(19) United States
(12) Patent Application Publication
(10) Pub. No.: US 2024/0052434 A1
(43) Pub. Date: Feb. 15, 2024

(54) TOMATO PLANTS HAVING FRUIT WITH HIGH ZEAXANTHIN CONTENT

(71) Applicant: Yissum Research Development Company of the Hebrew University of Jerusalem Ltd., Jerusalem (IL)

(72) Inventors: Joseph Hirschberg, Jerusalem (IL); Daniel Zamir, Gedera (IL); Uri Karniel, Jerusalem (IL); Amit Koch, Rehovot (IL)

(21) Appl. No.: 17/641,791

(22) PCT Filed: Sep. 9, 2020
(86) PCT No.: PCT/IL2020/050982

§ 371 Date: Mar. 9, 2022

Related U.S. Application Data
(60) Provisional application No. 62/897,411, filed on Sep. 9, 2019.

(51) Int. Cl. C12Q 1/6895; C12N 9/90; C12N 9/02; C12N 15/82

(52) U.S. Cl. CPC C12Q 1/6895; C12N 9/90; C12Y 505/01019; C12N 9/0073; C12Y 114/1309; C12N 9/0077; C12N 15/8245; C12Q 2600/13; C12Q 2600/156

(57) ABSTRACT

The present invention relates to tomato plants having fruit with elevated nutritional and health benefits, particularly fruit with elevated content of the carotenoid zeaxanthin, a carotenoid offering a range of health benefits, particularly thought to inhibit the progression of age-related macular degradation (AMD), the most prevalent cause of blindness in developed countries.

### FIELD OF THE INVENTION

The present invention relates to tomato plants having fruit with elevated nutritional and health benefits, particularly fruit with elevated content of the carotenoid zeaxanthin, a carotenoid offering a range of health benefits, particularly thought to inhibit the progression of age-related macular degradation (AMD), the most prevalent cause of blindness in developed countries.

### BACKGROUND OF THE INVENTION

Carotenoids are natural tetraterpenoid molecules that typically contain 40 carbon atoms made of 8 isoprene units. Carotenoids play essential functions in all photosynthetic organisms as accessory pigments participating in light harvesting in the photosynthetic reaction centers and in protecting the photosynthetic apparatus against oxygen radicals produced by excessive light energy. Carotenoids are synthesized in plants, algae, cyanobacteria and also in certain fungi and non-photosynthetic bacteria, but not in animals. Nonetheless, carotenoids are essential components in the human diet. Firstly, and most importantly, β-carotene, α-carotene and β-cryptoxanthin are the main precursors for vitamin A (retinol) and its derivatives 11-cis-retinal and all-trans-retinoic acid. Other health benefits of carotenoids are related to their properties as antioxidants. Carotenoids are implicated in reducing cancer and cardiovascular diseases, and in enhancing the immune system, bone health and cognitive functions (Rodriguez-Concepcion M et al., 2018. Prog. Lipid Res. 70, 62-93. doi: 10.1016/j.plipres.2018.04.004; Eggersdorfer M and Wyss A. 2018. Arch. Biochem. Biophys. 652, 18-26. doi: 10.1016/j.abb.2018.06.001).

The xanthophylls (oxygenated carotenoids) zeaxanthin, lutein and their metabolite meso-zeaxanthin, accumulate in the macula lutea of the human retina. In recent years, it has been recognized that these xanthophylls protect the macula by filtering damaging blue-light irradiation and through their antioxidant activity (Johnson E J. 2014. Nutr. Rev., 72, 605-612; Arunkumar R et al., 2018. Eye (Lond) 32, 992-1044; Sauer L et al., 2019. Annu. Rev. Nutr., 39:95-120). Deficiency of xanthophylls in humans leads to eye diseases and facilitates the development of age-related macular degeneration (AMD), the most prevalent cause of blindness in developed countries (Flaxman S R et al., 2017. Lancet Glob. Health., 5, e1221-e1234). Dietary supplementation of lutein and zeaxanthin increases their concentration in the macula and delays the progression of AMD by about 10% (the Age-Related Eye Disease Study 2 (AREDS2) JAMA 309, 2005-2015). Based on the AREDS2 nutritional recommendations, lutein and zeaxanthin are now the standard of care for reducing the probability of advanced AMD (Arunkumar et al., 2018, ibid; Wu J et al., 2015. JAMA Ophthalmol., 133, 1415-1424; Sauer et al., 2019, ibid). The importance of zeaxanthin in protecting the eye was emphasized following the recent discovery that it is the most concentrated xanthophyll in the center of the fovea. (Arunkumar, R et al., 2020. Biochim. Biophys. Acta Mol Cell Biol Lipids.,doi.org/10.1016/j.bbalip.2020.158617, 158617; Li B et al., 2020. Proc. Natl. Acad. Sci. U.S.A, 117(22), 12352-12358; Widomska J et al., 2020. Nutrients., 12, 1333). Other health benefits of zeaxanthin include anti-inflammatory effects (Johnson, E J. 2014. Nutr. Rev., 72, 605-612), reducing risk of atherosclerosis (Dwyer J H et al., 2001. Circulation, 103, 2922-2927), head and neck cancer (Leoncini E et al., 2016. Eur J Epidemiol. 31, 369-383) and breast cancer (Wang L et al., 2014. Br. J. Nutr., 111, 1686-1695). In spite of these benefits, zeaxanthin is not readily available in human diet. In green leafy vegetables, zeaxanthin is present in small or negligible amounts. Several orange-yellow fruits such as red peppers, oranges, papaya and mango, contain zeaxanthin at the range of 3-30 μg/g fresh weight (FW) (Dias M G et al., 2018. Agric. Food Chem., 66, 5055-5107; Holden J M et al., 1999. J. Food Comp. Analysis, 12, 169-196). Certain varieties of paprika, orange peppers and Goji berries (Lycium fruit) can reach high amounts of zeaxanthin (e.g., 600-800 μg/g dry weight) (Deli J et al., 1996. J Agr. Food. Chem., 44, 711-716; Garcia M I et al., 2007. Scientia Horticulturae, 113, 202-207; Hornero-Méndez D et al., 2002. Journal of agricultural food chemistry, 50, 5711-5716; Maiani G et al., 2009. Mol Nutr Food Res., 53 Suppl 2:S194-218., S194-S218; Niro S et al., 2017. Italian J. Food Sci., 29, 398-408; U.S. Pat. No. 9,247,694; Patsilinakos A et al., 2018. Food Chemistry, 268, 49-56; U.S. Pat. No. 9,247,694). However, these are high-cost minor foods for most consumers. Another source is sweet corn with about 5 μg/g FW zeaxanthin in regular varieties and up to 25 μg/g FW in kernels at eating-stage of bio-fortified varieties (O'Hare T J et al., 2014. Acta Horticdoi: 10.17660/ActaHortic.2014.1040.30; O'Hare T J et al., 2015. Arch. Biochem. Biophys. 572, 184-187. doi: 10.1016/j.abb.2015.01.015).

Tomato (Solanum lycopersicum) is globally the second largest vegetable crop with a yearly worldwide production over 170 million tons (FAO, 2017). Tomato fruit synthesize carotenoids that accumulate in chromoplasts, organelles adapted to store high concentration of carotenoids. Fresh tomato fruit contain therein 60-160 μg/g FW of the carotenoid lycopene, which constitutes 80-95 percent of the total carotenoid content. These attributes make tomato an excellent target crop of metabolic engineering to produce zeaxanthin.

Carotenoid biosynthesis in plants has been described at the molecular level (Ruiz-Sola M A and Rodriguez-Concepcion, M. 2012. Arabidopsis Book., 10:e0158.; Yuan H et al., 2015. Horticulture Research, 2, 15036; Rosas-Saavedra C and Stange, C. 2016. Subcell. Biochem., 79, 35-69. doi: 10.1007/978-3-319-39126-7_2; Moise A R et al., 2014. Chem. Rev. 114, 164-193). This process occurs within plastids where isopentenyl diphosphate (IPP) and its isomer dimethylallyl diphosphate (DMAPP), the precursors for plastidial isoprenoids, are synthesized de novo from pyruvate and D-glyceraldehyde 3-phosphate. Incorporation of three molecules of IPP with DMAPP gives rise to geranylgeranyl diphosphate (GGDP). Condensation of two GGDP molecules by the enzyme phytoene synthase (PSY) to produce phytoene marks the first committed step of the carotenoid synthesis pathway. Regulation of PSY has been shown to determine the flux of the pathway and thus affects the level of total carotenoids. Phytoene undergoes desaturation and cis-trans isomerization to give all-trans-lycopene. Cyclization of both ends of the linear molecule lycopene by lycopene β-cyclase (LCY-B) generates (β,β-carotene (β-carotene) whereas cyclization at one end by LCY-B and the other end by ε-cyclase (LCY-E) produces α-carotene (β,ε-carotene). α-carotene is converted to lutein by two hydroxylation steps carried out by two types of enzymes, β-ionone hydroxylases CHYB and CYP97A and the α-ionone hydroxylase CYP97C. Zeaxanthin is synthesized from β-carotene following hydroxylation of both rings by CHYB (FIG. 1). Three β-ionone hydroxylase enzymes exist in tomato, the non-haem iron BCH1 and BCH2, and the p450-type hydroxylase CYP97A. Zeaxanthin epoxidase (ZEP) converts zeaxanthin to violaxanthin that is metabolized to neoxanthin by a yet unidentified neoxanthin synthase (NSY).

The fleshy fruit of tomato develops from ovary over a period of 6-8 weeks after anthesis. At these stages, the fruit is green and its carotenoid composition is similar to that of leaves comprising mainly photosynthesis-associated xanthophylls. At the onset of ripening, so called the ‘breaker’ stage, lycopene begins to accumulate, mainly due to changes in gene expression. Transcription of genes for lycopene synthesis is upregulated and transcription of genes for lycopene cyclase enzymes that metabolize lycopene is decreased (Pecker et al., 1996. Plant Mol. Biol. 30, 807-819.; Ronen et al., 2000. Proc. Natl. Acad. Sci. USA, 97, 11102-11107; Ronen et al., 1999. Plant J. 17, 341-351; Bramley, 2002. J. Exp. Bot. 53, 2107-2113; Enfissi et al., 2017. Plant J. 89, 774-788). Expression of these genes is developmentally regulated but also affected by other factors, such as environment and hormones (Liu et al., 2015. Mol Plant. 8, 28-39; Klee and Giovannoni, 2011. Annu. Rev. Genet. 45, 41-59; Giovannoni et al., 2017. Ann. Rev. Plant Biol. 68, 61-84; Cruz et al., 2018. Front. Plant Sci. 9, 1370. doi.org10.3389/fpls.2018.01370; Li et al., 2019. New Phytol. 221, 1724-1741).

Several attempts to manipulate the expression of lycopene β-cyclase and β-carotene hydroxylase genes to facilitate xanthophyll accumulation in tomato fruit have been previously reported (D'Ambrosio et al., 2011. Transgenic Res. 20, 47-60; Enfissi et al., 2019. Plant Biotechnol. J. 17, 1501-1513; Giorio et al., 2008. Acta Hortic. 789, 277-284; Giuliano, 2014. Curr. Opin. Plant Biol. 19C, 111-117; Giuliano et al., 2008. Trends Biotechnol. 26, 139-145; Huang et al., 2013. Metabol. Eng. 17, 59-67; Nogueira et al., 2017. Proc. Natl. Acad. Sci. USA, 114, 10876-10881). Concurrent overexpression of carotene beta-hydroxylase 1(BCH1, CrtR-b1) and lycopene b-cyclase (LCYB) did not result in any appreciable increase in the xanthophyll content of ripe fruits compared to the overexpression of only the LCYB transgene (Giorio et al., 2008, ibid). Overexpression of carotene beta-hydroxylase 2 (BCH2, CrtR-b2) in tomato fruit produced free violaxanthin and a significant amount of esterified xanthophylls in hemizygous transgenic plants at the immature green stage (D'Ambrosio et al., 2011, ibid). Yet, at the ripe stage, total xanthophyll content was only 8% of total carotenoids with small amounts of zeaxanthin. Dharmapuri et al., (2002. FEBS Lett. 26068, 1-5) obtained ca. 13 μg/g FW of zeaxanthin plus β-cryptoxanthin in tomato fruit by simultaneous transgenic expression of Arabidopsis LCYB and pepper BCH (b-Chy) genes with the Pds promoter.

A publication of the inventors of the present invention, published after the priority date of the present invention, describes a tomato line, named ‘Xantomato’, having fruit accumulating zeaxanthin at a concentration of 39 μg/g fresh weight (or 577 μg/g dry weight), which comprised ca. 50% of total fruit carotenoids compared to zero in the wild type.

It is desirable to develop a low-cost and sustainable source of natural zeaxanthin in tomato, a widespread crop that serves the fresh market as well as the industrial production.

### SUMMARY OF THE INVENTION

The present invention provides tomato plants having fruit producing zeaxanthin in a significant amount, such that the health benefits of this carotenoid may be exerted when the tomato fruit are consumed within a regular, vegetable-containing diet of a subject.

The present invention is based in part on the unexpected discovery that a genetic combination of three distinct genes within the tomato genome provides for elevated amounts of zeaxanthin in the tomato fruit, reaching at least 20% of the total carotenoids produced by the fruit, typically at least 30% and up to more than 50% of the tomato fruit total carotenoid content. The genetic combination, not naturally present in tomato (Solanum lycopresicum) plants, comprises (i) a dominant allele of a chromoplast-specific lycopene β-cyclase, CycB termed HIGH-BETA allele (B)); (ii) a mutant allele of zeaxanthin epoxidase (ZEP) HIGH-PIGMENT 3 (hp3), the mutation impairing the conversion of zeaxanthin to violaxanthin; and the mutant green-stripe (gs) allele, a methylated isoform of TAGL1. Presence of an additional mutation within the tomato genome, the HIGH-PIGMENT mutant allele (high-pigment 2, hp2^{dg}), further contribute to elevation of the zeaxanthin content within the tomato fruit.

Unexpectedly, the present invention now shows that enhancing the hydroxylation of β-carotene, a necessary step in the production of zeaxanthin, by overexpression of β-carotene hydroxylase (BCH2) in a genetic background of hp3/B^{sh}, raised zeaxanthin accumulation, but to a lesser extent as compared to the hp3/B^{sh}/gs plants.

According to one aspect, the present invention provides a tomato plant having fruit with high zeaxanthin content, the tomato plant comprising within its genome a combination of (i) at least one HIGH-BETA (B) allele; (ii) at least one HIGH-PIGMENT 3 (hp3) allele, and (iii) at least one green-stripe (gs) allele, wherein the zeaxanthin content in the tomato fruit is at least 20% out of the total carotenoid content.

According to some embodiments, the tomato plant is homozygous for HIGH-BETA (B) allele. According to other embodiments, the tomato plant has a single allele of HIGH-BETA (B) allele.

According to some embodiments, the HIGH-BETA (B) allele is derived from Solanum habrochaites (B^{sh} allele).

According to certain embodiments, the B^{sh} allele comprises a polynucleotide encoding a lycopene β-cyclase, the polynucleotide comprising a nucleic acid sequence having at least 90% identity to SEQ ID NO:1 or a fragment thereof. According to certain embodiments, the B^{sh} allele comprises a promoter comprising nucleic acids 1-2335 of SEQ ID NO:1. According to some embodiments, the B^{sh} allele promoter comprises insertions of total of 43 bp compared to a wild type allele. According to certain embodiments, the B^{sh} allele comprises a polynucleotide marker amplified by a primer pair comprising a pair of oligonucleotides having a nucleic acid sequence selected from the group consisting of SEQ ID NO:2 and SEQ ID NO:3. According to certain embodiments, the marker comprises a nucleic acid sequence having at least 90% identity to the nucleic acid sequence set forth in SEQ ID NO:4. According to certain currently exemplary embodiments, the marker comprises the nucleic acid sequence set forth in SEQ ID NO:4.

According to additional or alternative embodiments, the B^{sh} allele comprises a polynucleotide marker amplified by a pair of primers comprising a pair of oligonucleotides having a nucleic acid sequence selected from the group consisting of SEQ ID NO:5 and SEQ ID NO:6, wherein the amplified marker comprises the nucleic acid sequence set forth in SEQ ID NO: 7.

According to certain embodiments, the hp3 allele encodes a mutant zeaxanthin epoxidase (ZEP), having a reduced or no capability to convert zeaxanthin to violaxanthin. According to some embodiments, the tomato plant is homozygous for the hp3 allele. According to certain embodiments, the mutant ZEP comprises an amino acid sequence having at least 90% identity to the amino acid sequence set forth is SEQ ID NO:8, wherein said sequence comprises lysine (K) at position 142. According to certain embodiments, the mutant ZEP is encoded by a polynucleotide having at least 90% identity to the nucleic acid sequence set forth in SEQ ID NO:9 wherein said sequence comprises the nucleotide adenine (A) at position 424. According to certain embodiments, the hp3 allele comprises a polynucleotide marker amplified by a pair of primers comprising a pair of oligonucleotides having a nucleic acid sequence selected from the group consisting of SEQ ID NO:10 and SEQ ID NO:11. According to certain currently exemplary embodiments, the marker comprises a nucleic acid sequence having at least 90% identity to the nucleic acid sequence set forth in SEQ ID NO:12, wherein the nucleic acid comprises the nucleotide adenine (A) at position 562.

According to certain exemplary embodiments, the hp3 allele comprises a marker having the nucleic acid sequence set forth in SEQ ID NO:12. According to certain embodiments, the tomato plant is homozygous for the gs allele.

According to certain embodiments, the gs allele comprises a polynucleotide marker amplified by a pair of primers comprising a pair of oligonucleotides having a nucleic acid sequence selected from the group consisting of SEQ ID NO:13 and SEQ ID NO:14, wherein the amplified marker comprises the nucleic acid sequence set forth in SEQ ID NO:15.

According to certain embodiments, the green-stripe (gs) allele is an allele originated from line LA0212 (Tomato Genetics Resource Center, Davis).

According to certain exemplary embodiments, the tomato plant comprises within its genome HIGH-BETA (B) homozygous alleles; (ii) HIGH-PIGMENT 3 (hp3) homozygous alleles, and (iii) green-stripe (gs) homozygous alleles.

According to some embodiments, the tomato plant further comprises within its genome at least one HIGH-PIGMENT 2 mutant allele (high-pigment 2). According to specific embodiments, the HIGH-PIGMENT2 mutant allele is hp2^{dg}. According to certain embodiments, the tomato further comprises hp2^{dg} homozygous alleles.

According to certain embodiments, the hp2^{dg} allele comprises a polynucleotide marker amplified by a primer pair comprising a pair of oligonucleotides having the nucleic acid sequence selected from the group consisting of SEQ ID NO:16 and SEQ ID NO:17. According to certain currently exemplary embodiments, the marker comprises a nucleic acid sequence having at least 90% identity to the nucleic acid sequence set forth in SEQ ID NO:18. According to certain exemplary embodiments, the marker comprises the nucleic acid sequence set forth in SEQ ID NO:18.

According to certain embodiments, the fruit of the tomato plant comprises at least 20%, at least 30%, at least 40%, at least 50%, at least 60% or more zeaxanthin out of the total carotenoid content.

According to some embodiments, the fruit carotenoid is selected from the group consisting of zeaxanthin, β-carotene, β-cryptoxanthin, lutein, and combinations thereof.

According to certain embodiments, the fruit of the tomato plant is essentially devoid of lycopene.

According to certain currently exemplary embodiments, the zeaxanthin content in the tomato fruit is at least 15 μg per gram of fruit fresh weight (FW). According to additional certain exemplary embodiments, the zeaxanthin content in the tomato fruit is at least 20, at least 25, at least 30, at least 35 or at least 40 μg/g fruit FW.

According to certain embodiments, the tomato plant having the allelic combination of the invention and producing fruit with high zeaxanthin content is a crop cultivar (cv).

Seeds, cuttings and any other plant parts that can be used for propagation, including isolated cells and tissue cultures are also encompassed within the scope of the present invention. It is to be understood that plants produced from said seeds or other propagating material comprise the unique allelic combination of the present invention, comprising at least the B^{sh}, hp3, and gs alleles, preferably further comprising the hp2^{dg} allele as described hereinabove, and produce tomato fruit with zeaxanthin content of at least 20% out of the total carotenoid content in the fruit.

According to certain embodiments, the present invention provides cell- and/or tissue cultures derived from the tomato plant of the invention or parts thereof, said cell—and/or tissue cultures produce zeaxanthin at an amount of at least 20% out of the total carotenoid content produced by said cell- and/or tissue cultures. According to certain embodiments, the total carotenoids and/or isolated zeaxanthin are then extracted from the cells/tissue or from the growth medium.

According to certain embodiments, the present invention provides tomato fruit having zeaxanthin content of at least 20% out of the total carotenoid content in the fruit.

According to certain exemplary embodiments, the fruit comprise at least 15 μg zeaxanthin per gram of said fruit FW. According to further exemplary embodiments, the fruit comprise at least 20 μg, at least 25 μg, at least 30 μg, at least 35 μg or more zeaxanthin per gram fruit FW.

According to some embodiments, the tomato plant is a transgenic plant.

According to a further aspect, the present invention provides a cell of tomato plant, the cell comprising within its genome a combination of (i) at least one HIGH-BETA (B) allele; (ii) at least one HIGH-PIGMENT 3 (hp3) allele, and (iii) at least one green-stripe (gs) allele.

According to a further aspect, the present invention provides a method for elevating the content of zeaxanthin in tomato fruit relative to the total carotenoid content in the fruit, comprising expressing within the genome of a tomato plant producing said fruit at least one of (i) at least one HIGH-BETA (B) allele; (ii) at least one HIGH-PIGMENT 3 (hp3) allele, (iii) at least one green-stripe (gs) allele or a combination thereof.

According to certain embodiments, the HIGH-BETA allele is B^{sh} According to certain embodiments, the tomato plant is devoid of the B allele, the hp3 allele, and the gs allele, and the method comprises expressing a combination of the three alleles.

According to certain embodiments, the tomato plant comprises one allele selected from the group consisting of the B allele, the hp3 allele, and the gs allele, and the method comprises expressing the other two alleles.

According to certain embodiments, the tomato plant comprises two alleles selected from the group consisting of the B allele, the hp3 allele, and the gs allele, and the method comprises expressing the third allele.

According to some embodiments, the method comprises expressing within the genome of the tomato plant two homozygous B alleles, two homozygous hp3 alleles, and two homozygous gs alleles.

According to certain embodiments, the method further comprises expressing within the genome of the tomato plant at least one HIGH-PIGMENT 2 mutant allele within the genome of the tomato plant. According to specific embodiments, the HIGH-PIGMENT 2 mutant allele is hp2^{dg}. According to certain embodiments, the method further comprises expressing two homozygous hp2^{dg} alleles.

The B^{sh} allele, hp3 allele, gs allele and hp2^{dg} allele are as described hereinabove.

Any method as is known in the art for expressing the alleles within the genome of tomato plants can be used according to the teachings of the present invention.

According to certain embodiments, the method comprises transforming into at least one cell of the tomato plant at least one DNA construct or expression vector comprising the B^{sh} allele, hp3 allele, gs allele, hp2^{dg} or a combination thereof. It is to be explicitly understood that each of the allele can be transformed by a separate DNA construct or expression vector, or a combination of alleles can be transformed by a single DNA construct or expression vector.

According to certain embodiments, the method comprises introducing by genome editing at least one allele selected from the group consisting of B^{sh} allele, hp3 allele, gs allele, hp2^{dg} and a combination thereof into at least one cell of the tomato plant.

According to certain embodiments, the tomato plant is a tomato cultivar.

According to yet another aspect, the present invention provides a method for producing a tomato plant having fruit with elevated content of zeaxanthin compared to a corresponding tomato plant, comprising the steps of:
(a) introducing into a recipient tomato plant at least one genetic element from at least one donor plant, the genetic element is selected from the group consisting of a genetic element comprising B allele; a genetic element comprising hp3 allele; and a genetic element comprising gs allele; thereby producing offspring tomato plants;
(b) examining a nucleic acid sample obtained from each offspring tomato plants for the presence of the at least one allele;
(c) selecting tomato plants comprising a combination of said B allele, hp3 allele and gs allele, thereby producing tomato plants having fruit with zeaxanthin content of at least 20% out of the total carotenoid content in the fruit.

According to certain exemplary embodiments, the method comprises introducing a combination of genetic elements comprising the B allele, hp3 allele and gs allele. The genetic elements can each originate from a different donor plant or a combination of genetic elements can originate from a single plant.

According to further exemplary embodiment, the method further comprises introducing a genetic element comprising ab hp2^{dg} allele, thereby selecting tomato plants comprising a combination of the B allele, hp3 allele, gs allele and hp2^{dg} allele.

According to certain embodiments, the B allele is B^{sh} allele. The alleles are as described hereinabove.

According to certain embodiments, the genetic element is introduced by crossing the donor tomato plant and the recipient tomato plant.

According to certain embodiments, the genetic element in isolated from the donor plant and introduced to the recipient plant by transformation or genome editing as is known in the art and as described hereinbelow.

According to certain embodiments, examining the nucleic acid sample obtained from each offspring tomato plants for the presence of the at least one allele comprises identifying at least one nucleic acid marker indicative of said allele. The markers are as described hereinabove.

According to certain embodiments, the recipient tomato plant is a tomato cultivar.

According to some embodiments, the tomato cultivar is an elite cultivar.

It is to be understood that any combination of each of the aspects and the embodiments disclosed herein is explicitly encompassed within the disclosure of the present invention.

Other objects, features and advantages of the present invention will become clear from the following description and drawings.

### BRIEF DESCRIPTION OF THE FIGURES

FIG. 1 presents the biosynthetic steps in the conversion of lycopene to zeaxanthin in tomato. CycB, chromoplastic lycopene β-cyclase; CrtR-b (ChyB), non-heme β-carotene hydroxylase; CrtZ, bacterial β-carotene hydroxylase; CYP97B, cytochtrome p450β-carotene hydroxylase; LcyB, chloroplastic lycopene β-cyclase.

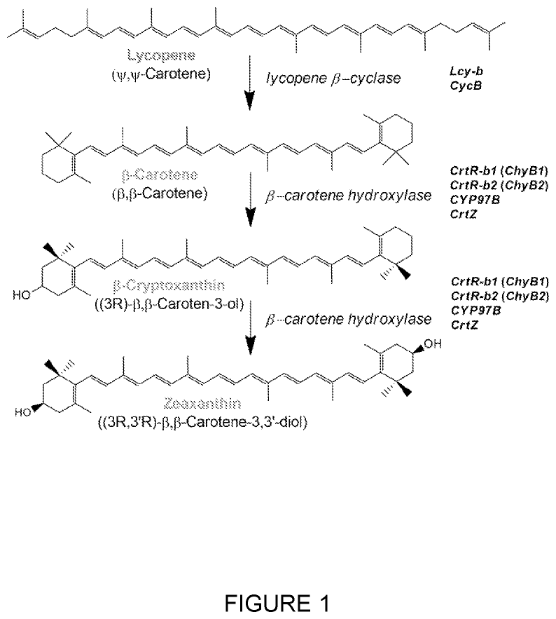

FIG. 2 shows the expression of carotenoid biosynthesis genes in ripe fruit (breaker+3 days) of the Wild Type (M82) and high-beta (B^{sh}) lines.

FIG. 3 shows concentration of zeaxanthin+β-cryptoxanthin (μg/g FW) in ripe fruits from various genotypes.

FIG. 4 shows carotenoids in E. coli cells expressing BCH2 from C. Clementina.

FIG. 5 shows concentration of zeaxanthin plus β-cryptoxanthin in ripe fruits of transgenic lines expressing the C. clementina BCH2 (μg/g FW).

FIG. 6 shows carotenoid composition (μg/gr Fruit FW) in fruits of a progeny of the cross between hp3/B^{sh}/gs and hp3/B^{sh}/gs/hp2^{dg}.

### DETAILED DESCRIPTION OF THE INVENTION

The present invention provides tomato fruit, and tomato plant producing same, the fruit containing elevated content of zeaxanthin, a carotenoid known to have beneficial effects of mammalian health, particularly in maintaining good eye vision and in preventing light-induced oxidative damage that could lead to macular degeneration (AMD).

The present invention shows that carotenoid biosynthesis in tomato fruit can be successfully manipulated to achieve zeaxanthin accumulation at a concentration of about μg/g fruit FW, which comprises more than 50% of the total fruit carotenoids. This zeaxanthin concentration is hitherto not known to be reached in a major crop such as a tomato. Furthermore, such high zeaxanthin amount in tomato fruit significantly increases zeaxanthin availability via the human diet, as tomatoes (fresh and/or processed) are regularly consumed by a variety of populations around the world, and most of the zeaxanthin is in its free from, a form having better bioavailability compared to esterified zeaxanthin. The tomato fruit high-zeaxanthin is attributed to the unique combination of three primary mutated alleles, BETA (B^{sh}), high-pigment 3 (hp3) and green-stripe (gs) within the genome of tomato plants demonstrated herein for the first time.

### Definitions

The terms “comprise”, “comprising”, “includes”, “including”, “having” and their conjugates mean “including but not limited to”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “zeaxanthin” refers to a pigment belonging to the xanthophyll division of carotenoids. It is also known as β,β-carotene-3,3′-diol and have the IUPAC name 4-[18-(4-hydroxy-2,6,6-trimethyl-1-cyclohexenyl)-3,7,12,16-tetramethyl-octadeca-1,3,5,7,9,11,13,15,17-nonaenyl]-3,5,5-trimethyl-cyclohex-3-en-1-ol.

As used herein, the term “allele” refers to any of one or more alternative form of a gene, all of which alleles relates to one trait or characteristic. In a diploid cell or organism, the two alleles of a given gene occupy corresponding loci on a pair of homologous chromosomes.

As used herein, the terms “tomato” and “tomato plant” are used herein to refer to any plant, line or population of the Solanaceae clade Lycopersicon, including but not limited to Solanum lycopersicum, Solanum lycopersicum var. cerasiforme, Solanum cheesmaniae, Solanum chilense, Solanum chmielewskii, Solanum habrochaites, Solanum neorickii, Solanum pennellii, Solanum ′N/peruvianum′, Solanum lycopersicum, Solanum juglandifolium, Solanum ochranthum, Solanum sitiens, or Solanum lycopersicoides. Solanum lycopersicum is a diploid species with a haploid set of 12 chromosomes.

It is to be noted that the newly proposed scientific name for the modern (cultivated) tomato is Solanum lycopersicum.

The term “HIGH-BETA” refers to an allele of a gene encoding lycopene β-cyclase mutant with high activity. According to certain exemplary embodiments, the genomic sequence of the lycopene β-cyclase mutant gene is set forth in SEQ ID NO:1. According to certain embodiments, the “HIGH-BETA allele” refers to the allele present in the green-fruited wild species Solanum habrochaites (LA0316) (Dalal M et al., 2010. BMC. Plant Biol., 10:61; The C. M. Rick Tomato Genetics Resource Center, TGRC, tgrc.ucdavis.edu/Data/Acc/GenDetail.aspx?Gene=b), termed herein “B^{sh}”,

The terms “HIGH-PIGMENT 3” and “hp3” are used herein interchangeably and refer to an allele of a gene encoding a mutant zeaxanthin epoxidase (ZEP), having a reduced or no capability to convert zeaxanthin to violaxanthin. According to certain embodiments, the ZEP mutant is encoded by a nucleic acid sequence comprising the nucleic acid sequence set forth in SEQ ID NO:9, having the amino acid sequence set forth in SEQ ID NO:8.

The terms “green-stripe” and “gs” are used herein interchangeably and refers to a naturally mutated allele present in a variety of tomato. Recently, it has been reported that the GS locus encodes a MADS-box transcription factor named TAGL1. Low degrees of methylation of the TAGL1 promoter led to light green stripes in tomato plants having the mutated gs gene (Liu G et al., 2020. New Phytologist doi: 10.1111/nph.16705). The gs allele exists in many different tomato lines, available from the Tomato Genetics Resource Center (TGRC), Davis, CA.

According to certain embodiments, the gs allele comprises a polynucleotide marker amplified by a pair of primers comprising a pair of oligonucleotides having a nucleic acid sequence selected from the group consisting of SEQ ID NO:13 and SEQ ID NO:14, wherein the amplified marker comprises the nucleic acid sequence set forth in SEQ ID NO:15. According to certain embodiments, digesting the marker with the restriction enzyme EcoRI results in the formation of two fragments. According to certain embodiments, digestion of the marker results in a fragment having 59 base-pairs (bp) and a fragment having 355 bp.

According to certain exemplary embodiments, the term refers to an allele of a locus mapped to chromosome #7 of tomato originated from line LA0212 (tgrc.ucdavis.edu/Data/Acc/GenRepeater.aspx?Gene=gs).

The terms “HIGH-PIGMENT 2” and “hp2” are used herein interchangeably and refer to an allele of the tomato homolog of Detl in Arabidopsis (Mustilli, A. C., et al. 1999. Plant Cell, 11, 145-157). According to certain exemplary embodiments, the term refers to hp2^{dg}. Tomato lines harboring this allele may be available from the Tomato Genetics Cooperative (TGC; tgc.ifas.ufl.edu/vol55/vol55html/vol55stocks.htm). According to certain embodiments, the hp2^{dg} allele comprises a polynucleotide marker having the nucleic acid sequence set forth in SEQ ID NO:18.

The term “gene”, as used herein, refers to a hereditary unit consisting of a sequence of DNA that occupies a specific location on a chromosome and that contains the genetic instruction for a particular characteristics or trait in an organism. The term “gene” thus refers to a nucleic acid (e.g., DNA or RNA) sequence that comprises coding sequences necessary for the production of RNA or a polypeptide or its precursor. A functional polypeptide can be encoded by a full-length coding sequence or by any part thereof as long as the desired activity or functional properties (for example, enzymatic activity, ligand binding, signal transduction, etc.) of the polypeptide are retained. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene. The term “gene” encompasses both cDNA and genomic forms of a gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

As used herein, the term “allele” refers to alternative or a variant form of a gene or of any kind of identifiable genetic element, which are alternative in inheritance because they are situated at the same locus in homologous chromosomes. Such alternative or variant forms may be the result of single nucleotide polymorphisms, insertions, inversions, translocations or deletions, or the consequence of gene regulation caused by, for example, chemical or structural modification, transcription regulation or post-translational modification/regulation. In a diploid cell or organism, the two alleles of a given gene or genetic element typically occupy corresponding loci on a pair of homologous chromosomes.

The term “genotype” as used herein refers to the genetic constitution of a cell or organism. As is known in the art, a genotype can relate to a single locus or to multiple loci, whether the loci are related or unrelated and/or are linked or unlinked. In some embodiments, an individual plant genotype relates to one or more genes that are present in the genome of the plant wherein the one or more of the genes are involved in the expression of a phenotype of interest (e.g. elevated content of fruit zeaxanthin as defined herein). According to certain aspects of the present invention the tomato genotype comprises a combination of at least one HIGH-BETA (B^{sh}) allele; at least one HIGH-PIGMENT 3 (hp3) allele, and at least one green-stripe (gs) allele.

As used herein, the term “breeding”, and grammatical variants thereof, refer to any process that generates a progeny individual. Breeding can be sexual or asexual, or any combination thereof. Exemplary non-limiting types of breeding include crossings, selfing, doubled haploid derivative generation, and combinations thereof.

As used herein the term “selfing” refers to a controlled self-pollination of a plant, i.e. contacting pollen and ovule produced by the same plant. The term “crossing” refers to controlled cross-pollination, i.e. contacting pollen and ovule each produced by a different plant.

As used herein, the term “donor” with reference to a plant relates to a plant or plant line from which the trait, allele, or genomic segment originates, and which donor may have the trait, allele, or genomic segment either heterozygous or homozygous.

The terms “recipient” and “target with reference a plant are used herein interchangeably and relate to a plant or plant line receiving the trait, allele, or genomic segment from a donor, and which recipient may or may not have the trait, allele, or genomic segment itself either heterozygous or homozygous.

The term “genetically modified plant” refers to a plant comprising at least one cell genetically altered by man. The genetic modification includes modification of an endogenous gene(s), for example by introducing mutation(s) deletions, insertions, transposable element(s) and the like into an endogenous polynucleotide or gene of interest. Additionally, or alternatively, the genetic modification includes transforming the organism cell with heterologous polynucleotide to produce transgenic organism.

As used herein, the term “heterologous polynucleotide” refers to a nucleic acid sequence which is not naturally expressed within the plant (e.g., a nucleic acid sequence from a different species, or, according to certain embodiments of the present invention, from a different tomato line, cultivar or variety). The heterologous polynucleotide may be introduced into the plant in a stable or transient manner, so as to produce a ribonucleic acid (RNA) molecule and/or a polypeptide molecule. The term “endogenous” as used herein refers to a polynucleotide or polypeptide which is naturally present and/or naturally expressed within a plant or a cell thereof.

A “cultivated tomato plant” or “tomato cultivar” or “tomato cultivar plant” is understood within the scope of the invention to refer to a plant of Solanum lycopersicum that is no longer in the natural state but has been developed by human care and for human use and/or consumption. “Cultivated tomato plants” or “tomato cultivars” or “tomato cultivar plants” are further understood to exclude those wild species which comprise the trait being subject of this invention as a natural trait and/or part of their natural genetics.

The term “heterozygous” is used herein to refer to unlike alleles at one or more corresponding loci on homologous chromosomes.

The term “homozygous” is used herein to refer to like alleles at one or more corresponding loci on homologous chromosomes.

Tomato fruit contain high concentration of lycopene as the major carotenoid pigment that accumulates during ripening due to its enhanced synthesis and reduced metabolism to cyclic carotenes. These metabolic processes are mainly regulated by differential gene expression. Several attempts to manipulate the expression of lycopene β-cyclase and β-carotene hydroxylase genes in order to obtain xanthophyll accumulation in tomato fruit have been previously reported. Concurrent over-expression of carotene beta-hydroxylase 1 (BCH1, CrtR-b1) and lycopene β-cyclase (LCYB) did not result in any appreciable increase in the xanthophyll content of ripe fruit compared to the over-expression of only the LCYB transgene (Giorio G et al., 2008. ibid). Over-expression of carotene beta-hydroxylase 2 (BC-12, CrtR-b2) in tomato fruit produced free violaxanthin and significant amount of esterified xanthophylls in hemizygous transgenic plants at the immature green stage (D'Ambrosio C et al., 2011. Transgenic Res., 20, 47-60). Yet, at the ripe stage, total xanthophyll content was only 8% of total carotenoids with negligible amounts of zeaxanthin (D'Ambrosio C et al., 2011, ibid). In another experiment, simultaneous expression in tomato fruit of Arabidopsis LCYB and pepper BCH (b-Chy) genes with the Pds promoter resulted in accumulation of about 13 μg/g FW of zeaxanthin plus β-ciyptoxanthin (Dharmapuri S et al., 2002, ibid).

According to certain aspects, the present invention provides a tomato plant having fruit with high zeaxanthin content, the tomato plant comprising within its genome a combination of (i) at least one HIGH-BETA (B) allele; (ii) at least one HIGH-PIGMENT 3 (hp3) allele, and (iii) at least one green-stripe (gs) allele, wherein the zeaxanthin content in the tomato fruit is at least 20% out of the total carotenoid content.

According to certain aspects, the present invention provides a tomato plant having fruit with high zeaxanthin content, the tomato plant comprising within its genome a combination of (i) at least one HIGH-BETA (B^{sh}) allele; (ii) at least one HIGH-PIGMENT 3 (hp3) allele, and (iii) at least one green-stripe (gs) allele, wherein the zeaxanthin content in the tomato fruit is at least 20% out of the total carotenoid content.

According to certain aspects, the present invention provides a tomato seed comprising within its genome a combination of (i) at least one HIGH-BETA (B) allele; (ii) at least one HIGH-PIGMENT 3 (hp3) allele, and (iii) at least one green-stripe (gs) allele, wherein a tomato plant grown from the seed produces fruit with zeaxanthin content of at least 20% out of the total carotenoid content.

According to certain aspects, the present invention provides a tomato fruit having zeaxanthin content of at least 20% out of the total carotenoid content in the fruit.

According to certain embodiments, the tomato fruit of the present invention comprises at least 25%, at least 30%, at least 35%, at least 40%, at least 45%, at least 50%, at least 55%, at least 60%, or at least 75% zeaxanthin out of the total carotenoid content in the fruit. Each possibility represents a separate embodiment of the present invention.

According to certain embodiments, the tomato fruit comprises from about 20% to about 75% zeaxanthin out of the total carotenoid content in the fruit. According to certain embodiments, the tomato fruit comprises from about 20% to about 60% zeaxanthin out of the total carotenoid content in the fruit. According to certain embodiments, the tomato fruit comprises from about 20% to about 50% zeaxanthin out of the total carotenoid content in the fruit.

According to certain exemplary embodiments, the fruit comprise at least 15 μg zeaxanthin per gram of said fruit FW. According to further exemplary embodiments, the fruit comprise at least 20 μg, at least 25 μg, at least 30 μg, at least 35 μg, at least 40 μg at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg or more zeaxanthin per gram fruit FW. Each possibility represents a separate embodiment of the present invention.

According to some embodiments, the tomato fruit of the present invention comprise from about 15 μg zeaxanthin per gram fruit FW to about 60 μg zeaxanthin per gram fruit FW. According to some embodiments, the tomato fruit comprise from about 15 μg zeaxanthin/g fruit FW to about 50 μg zeaxanthin/g fruit FW. According to some embodiments, the tomato fruit comprise from about 20 μg zeaxanthin/g fruit FW to about 50 μg zeaxanthin/g fruit FW.

High levels of β-carotene in tomato fruit occur in the mutant BETA where the fruit-specific lycopene β-cyclase (CycB) gene is highly expressed (Ronen et al., 2000, ibid). All known HIGH BETA (B) alleles were originated from green-fruited wild tomato species by genetic introgression of (Lincoln R E and Porter J W. 1950. Genetics, 35, 206-211; Tomes M L et al., 1958. Botanical Gazzete, 1264, 250-253; Tomes M L et al., 1954. Genetics, 39, 810-817; Stommel, J R 2001. Hortscience, 36, 387-388; Stommel J R and Haynes, K G. 1994. J. Hered., 85, 401-404; Ronen et al., 2000, ibid). In the present invention, the allele B^{sh} originated from S. habrochaites was used because it displays the strongest phenotype among known BETA mutations as evident by the fact that β-carotene in its fruit represents >80% of total carotenoids (Table 3). This reflects a highly efficient lycopene cyclization activity by CYCB encoded by B^{sh}. Converting β-carotene to downstream xanthophylls was found to be difficult to attain. Previous studies have suggested that the β-carotene accumulated naturally in BETA fruit is not accessible to hydroxylation (Giuliano, G., et al. 2008. Trends Biotechnol., 26, 139-145).

According to certain embodiments, the B^{sh} allele comprises a polynucleotide encoding a lycopene β-cyclase, the polynucleotide comprising a nucleic acid sequence having at least 90%, at least 91%, at least 92%, at least 93%, at least 94%, at least 95%, at least 96%, at least 97%, at least 98%, or at least 99% identity to SEQ ID NO:1 or a fragment thereof. Each possibility represents a separate embodiment of the present invention. According to certain embodiments, the B^{sh} allele comprises the nucleic acid sequence set forth in SEQ ID NO:1 or a fragment thereof. According to certain embodiments, the fragment comprises a promoter. According to certain exemplary embodiments, the promoter comprises nucleic acids 1-2335 of SEQ ID NO: 1. According to some embodiments, the B^{sh} allele promoter comprises three insertions, comprising total of 43 bp absent in a wild type allele. According to certain embodiments, the B^{sh} allele comprises a polynucleotide marker amplified by a primer pair comprising a pair of oligonucleotides having the nucleic acid sequence set forth in SEQ ID NO:2 and SEQ ID NO:3. According to certain embodiments, the marker comprises a nucleic acid sequence having at least 90%, 91%, at least 92%, at least 93%, at least 94%, at least 95%, at least 96%, at least 97%, at least 98% or at least 99% identity to the nucleic acid sequence set forth in SEQ ID NO:4. Each possibility represents a separate embodiment of the present invention. According to certain embodiments, the marker comprises the nucleic acid sequence set forth in SEQ ID NO:4.

According to additional or alternative embodiments, the B^{sh} allele comprises a polynucleotide marker amplified by a pair of primers comprising a pair of oligonucleotides having a nucleic acid sequence selected from the group consisting of SEQ ID NO:5 and SEQ ID NO:6, wherein the amplified marker comprises the nucleic acid sequence set forth in SEQ ID NO:7. According to certain exemplary embodiments, the marker comprises the nucleotide Adenine (A) at position 91 compared to Guanine (G) present in a marker amplified by the pair of primers comprising SEQ ID NO:5-6 in a genetic material obtained from a wild type tomato.

According to certain embodiments, the tomato plant is homozygous for the B^{sh}allele.

As is demonstrated in Table 3, the total carotenoid concentration in fruit of plants carrying B^{sh} is lower compared to that found in wild type tomato plants or cultivars (e.g. M82). The steady-state concentration of carotenoids in fruit is controlled by the flux of the biosynthetic pathway; by sequestration mechanisms that determines the storage capacity; and by degradation processes catalyzed by carotenoid cleavage dioxygenases (CCD). Loss-of-function of zeaxanthin epoxidase (ZEP) in the mutant hp3, or silencing of the ZEP gene, cause a modest buildup of zeaxanthin in fruit (Galpaz et al., 2008, ibid; Wolters, A. M., et al. 2010. Plant Mol. Biol., 73, 659-671; Romer et al., 2002. Metabolic Engineering, 4, 263-272; and Table 3). This phenomenon shows that β-carotene hydroxylation does take place in tomato fruit. Since zeaxanthin does not accumulate in either normal or in HIGH-BETA fruit, it has been deduced that it is metabolized to violaxanthin, which is the precursor for abscisic acid (ABA) synthesis. In addition, it is probable that fruit chromoplasts do not possess a mechanism to store free zeaxanthin and thus it can be constantly degraded by CCDs. Both processes may explain the lower total carotenoids in fruit of B^{sh}-carrying plants.

According to certain embodiments, the hp3 allele encodes a mutant zeaxanthin epoxidase (ZEP), having a reduced or no capability to convert zeaxanthin to violaxanthin. According to certain embodiments, the mutant ZEP comprises an amino acid sequence having at least 90%, at least 91%, at least 92%, at least 93%, at least 94%, at least 95%, at least 96%, at least 97%, at least 98%, or at least 99% identity to the amino acid sequence set forth is SEQ ID NO:8, wherein said sequence comprises lysine (K) at position 142. According to certain embodiments, the mutant ZEP comprises the amino acid sequence set forth in SEQ ID NO:8. Each possibility represents a separate embodiment of the present invention.

According to certain embodiments, the mutant ZEP is encoded by a polynucleotide having at least 90%, at least 91%, at least 92%, at least 93%, at least 94%, at least 95%, at least 96%, at least 97%, at least 98%, or at least 99% identity to the nucleic acid sequence set forth in SEQ ID NO:9 wherein said sequence comprises the nucleotide adenine (A) at position 424. Each possibility represents a separate embodiment of the present invention. According to certain embodiments, the mutant ZEP is encoded by a polynucleotide having the nucleic acid sequence set forth in SEQ ID NO:9. According to certain embodiments, the hp3 allele comprises a polynucleotide marker amplified by a primer pair comprising a pair of oligonucleotides having the nucleic acid sequence selected from the group consisting of SEQ ID NO:10 and SEQ ID NO:11. According to certain currently exemplary embodiments, the marker comprises a nucleic acid sequence having at least 90%, at least 91%, at least 92%, at least 93%, at least 94%, at least 95%, at least 96%, at least 97%, at least 98%, or at least 99% identity to the nucleic acid sequence set forth in SEQ ID NO:12, wherein the nucleic acid comprises the nucleotide A at position 562. Each possibility represents a separate embodiment of the present invention. According to certain exemplary embodiments, the hp3 allele comprises a marker having the nucleic acid sequence set forth in SEQ ID NO:12.

According to some embodiments, the tomato plant is homozygous for the hp3 allele. While plants with a combination of B^{sh} and hp3 yielded fruit with 7.7 μg/g⁻¹ fresh weight of zeaxanthin (Table 3), addition of the green-stripe (gs) alleles more than doubled this concentration, and the fraction of zeaxanthin plus β-cryptoxanthin from total β-cyclized carotenoids increased from about 21% to about 40% (Table 3). The GS locus, a methylated isoform of TAGL1 regulating diversified chloroplast development and carotenoid accumulation has been recently identified. Non-uniform pigmentation of fruit produced by GS was highly associated with methylation of the TAGL1 promoter, which is linked to an SNP on chromosome 7. High degrees of methylation of the TAGL1 promoter downregulated its expression, leading to green stripes; low degrees of methylation led to light green stripes in gs s (Liu et al., 2020, ibid).

The gs allele exists in numerous different tomato lines, available from TGRC (tgrc.ucdavis.edu/Data/Acc/GenRepeater.aspx?Gene=gs).

According to certain embodiments, the gs allele comprises a polynucleotide marker amplified by a pair of primers comprising a pair of oligonucleotides having a nucleic acid sequence selected from the group consisting of SEQ ID NO:13 and SEQ ID NO:14, wherein digesting the marker with the restriction enzyme EcoRI results in the formation of two fragments. According to certain embodiments, the gs allele polynucleotide marker comprises the nucleic acid sequence set forth in SEQ ID NO:15. According to certain embodiments, digestion of the marker results in a fragment having 59 base-pairs (bp) and a fragment having 355 bp.

According to certain embodiments, the gs allele is an allele originated from line LA0212.

The present invention further demonstrates that additional zeaxanthin accumulation was provided by adding to the B^{sh}/hp3/gs genotype the allelic mutation high-pigment 2 (hp2), which impairs the tomato DET1 homolog that functions in photomorphogenesis (Mustilli A C et al., 1999. ibid) and enhances fruit metabolites (Liu L et al., 2004. Proc. Natl. Acad. Sci. U.S.A, 101, 9897-9902). Fruit of allele hp2^{dg} of this mutation were found to be more active metabolically (Liu L et al., 2004, ibid; Levin I et al., 2006. Israel J. Plant Sci., 54, 179-190; Bino R J et al., 2005. New Phytol., 166, 427-438; Kolotilin I et al., 2007. Plant Physiology, 145, 389-401). The combination of alleles hp3, B^{sh}, gs and hp2^{dg}, not only elevated the level of zeaxanthin but also raised its ratio to at least about 50% out of total fruit carotenoids.

According to certain embodiments, the hp2^{dg} allele comprises a polynucleotide marker amplified by a primer pair comprising a pair of oligonucleotides having the nucleic acid sequence selected from the group consisting of SEQ ID NO:16 and SEQ ID NO:17. According to certain currently exemplary embodiments, the marker comprises a nucleic acid sequence having at least 90%, at least 91%, at least 92%, at least 93%, at least 94%, at least 95%, at least 96%, at least 97%, at least 98%, or at least 99% identity to the nucleic acid sequence set forth in SEQ ID NO:18. Each possibility represents a separate embodiment of the present invention. According to certain currently exemplary embodiments, the marker comprises the nucleic acid sequence set forth in SEQ ID NO:18.

Xanthophylls in chromoplast of flowers are usually sequestered as esters (Yamamizo C et al., 2010. Experimental Botany, 61, 709-719; Ariizumi T et al., 2014. The Plant Journal, 79, 453-465). All the zeaxanthin molecules accumulated in fruit in the plants of the present invention were in the free form, i.e. non-esterified. It is likely that this feature limits the storage capacity of zeaxanthin in tomato chromoplast and makes it more susceptible to enzymatic degradation. However, free carotenoids have a better bioavailability in a mammalian, particularly human body compared to the esterified form, and thus the tomato fruit of the present invention not only provide zeaxanthin at high amounts, the zeaxanthin is readily available and thus can exert its health beneficial effects.

According to certain embodiments, the tomato fruit of the present invention is essentially devoid of lycopene. As used herein, the term “essentially devoid” with reference to lycopene refers to lycopene concentration of up to 0.1% of the total carotenoid content, typically up to 0.01% of the total carotenoid content.

Transgenic expression of BCH2 from C. clementina in the genetic background of hp3/B^{sh} raised zeaxanthin accumulation, but significantly decreased the β-carotene, as well as the total carotenoids content. Accordingly, while hp3/B^{s}h/BCH2 plants showed elevated amounts of zeaxanthin compared to its content in wild type plants/cultivars, the amounts observed in B^{Sh}/hp3 gs and B^{Sh}/hp3gs/hp2dg have not yet been reached.

According to a further aspect, the present invention provides a method for elevating the content of zeaxanthin in tomato fruit relative to the total carotenoid content in the fruit, comprising expressing within the genome of a tomato plant producing said fruit a combination of (i) at least one HIGH-BETA (B^{sh}) allele; (ii) at least one HIGH-PIGMENT 3 (hp3) allele, and (iii) at least one green-stripe (gs) allele.

According to certain embodiment, the method further comprises expressing at least one HIGH-PIGMENT mutant allele (high-pigment 2, hp2^{dg}) within the genome of the tomato plant.

Introducing the combination of alleles resulting in the production of high-zeaxanthin tomato fruit into a tomato plant or a part thereof can be performed by any method as is known to a person skilled in the art.

According to certain embodiments, isolated polynucleotide or polynucleotides, typically in a form of a DNA construct comprising the nucleic acid sequences of the allele or alleles of the present invention can be introduced into the genome of a tomato plant to produce a tomato plant having the B^{sh} hp3 gs or B^{sh}/hp3/gs hp2^{dg} genotypes of the invention.

According to the teachings of the present invention, such a DNA construct comprises a nucleic acid sequence that comprises at least one allele of the invention. Typically, the DNA construct comprises each allele, or a tandem of alleles under control of, or operatively linked to, a regulatory element. According to certain embodiments, the regulatory element is selected from the group consisting of a promoter, an enhancer and a translation termination sequence. The DNA construct may contain one or more such operably linked gene/allele/regulatory element combinations. The construct(s) may be in the form of a plasmid, and can be used, alone or in combination with other plasmids, in a method for producing transgenic plants that produce fruit with the elevated zeaxanthin phenotype, using transformation methods known in the art to be suitable for transforming nucleic acid sequences into tomato (dicotyledonous) plants.

The DNA construct can include at least one marker (reporter) gene, operably linked to a regulatory element (such as a promoter) that allows transformed cells containing the marker to be either recovered by negative selection (by inhibiting the growth of cells that do not contain the selectable marker gene), or by positive selection (by screening for the product encoded by the markers gene). Many commonly used selectable marker genes for plant transformation are known in the art, and include, for example, genes that code for enzymes that metabolically detoxify a selective chemical agent which may be an antibiotic or an herbicide, or genes that encode an altered target which is insensitive to the inhibitor. Several positive selection methods are known in the art, such as mannose selection. Alternatively, marker-less transformation can be used to obtain plants without mentioned marker genes, the techniques for which are known in the art.

Methods for transforming a plant cell with nucleic acid sequences according to the present invention are known in the art. As used herein the term “transformation” or “transforming” describes a process by which a foreign nucleic acid sequence, such as a DNA construct, including a vector, enters and changes a recipient cell into a transformed, genetically modified or transgenic cell. Transformation may be stable, wherein the nucleic acid sequence is integrated into the plant genome and as such represents a stable and inherited trait, or transient, wherein the nucleic acid sequence is expressed by the cell transformed but is not integrated into the genome, and as such represents a transient trait. According to typical embodiments the nucleic acid sequence(s) of the present invention is stably transformed into a plant cell.

There are various methods of introducing foreign genes into both monocotyledonous and dicotyledonous plants (for example, Potrykus I. 1991. Annu Rev Plant Physiol Plant Mol Biol 42:205-225; Shimamoto K et al., 1989. Nature 338:274-276).

The principal methods of the stable integration of exogenous DNA into plant genomic DNA includes two main approaches: Agrobacterium-mediated gene transfer: The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation (Horsch et al., 1988. Plant Molecular Biology Manual A5, 1-9, Kluwer Academic Publishers, Dordrecht). A supplementary approach employs the Agrobacterium delivery system in combination with vacuum infiltration. Agrobacterium mediated transformation protocols for tomato plants are known to a person skilled in the art.

Direct nucleic acid transfer: There are various methods of direct nucleic acid transfer into plant cells. In electroporation, protoplasts are briefly exposed to a strong electric field, opening up mini-pores to allow DNA to enter. In microinjection, the nucleic acid is mechanically injected directly into the cells using micropipettes. In microparticle bombardment, the nucleic acid is adsorbed on microprojectiles such as magnesium sulfate crystals or tungsten particles, and the microprojectiles are physically accelerated into cells or plant tissues. Another method for introducing nucleic acids to plants is via the sonication of target cells. Alternatively, liposome or spheroplast fusion has been used to introduce expression vectors into plants.

Following transformation of tomato target tissues, expression of the above described selectable marker genes allows for preferential selection of transformed cells, tissues and/or plants, using regeneration and selection methods now well known in the art.

According to some embodiments of the invention, introducing the at least one allele of the invention is performed by means of genome editing.

Genome editing is a reverse genetics method which uses artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Genome editing is a powerful tool to impact target traits by modifications of the target plant genome sequence. Such modifications can result in new or modified alleles or regulatory elements.

According to certain exemplary embodiments, genome editing is used to modify the genome of a target tomato plant by introducing at least one allele of the invention to a desired genomic location.

According to yet another aspect, the present invention provides a method for producing a tomato plant having fruit with elevated content of zeaxanthin compared to a corresponding tomato plant, comprising the steps of:
(d) introducing at least one genetic element selected from the group consisting of: a genetic element comprising B^{sh} allele; a genetic element comprising hp3 allele; and a genetic element comprising gs allele; from at least one donor tomato plant into a recipient tomato plant, to produce offspring tomato plants;
(e) examining a nucleic acid sample obtained from each offspring tomato plants for the presence of the at least one allele;
(f) selecting tomato plants comprising said at least one B allele, hp3 and allele gs thereby producing tomato plants having fruit with zeaxanthin content of at least 20% out of the total carotenoid content in the fruit.

According to certain exemplary embodiments, the method comprises introducing a combination of genetic elements comprising the B allele, hp3 allele and gs allele. The genetic elements can each originate from a different donor plant or a combination of genetic elements can originate from a single plant.

According to further exemplary embodiment, the method further comprises introducing an hp2^{dg} allele, thereby selecting cultivated tomato plants comprising the B allele, hp3 allele, gs allele and hp2^{dg} allele.

According to certain embodiments, the B allele is B^{sh} allele. The alleles are as described hereinabove.

According to certain embodiments, the genetic element is introduced by crossing the donor plant and the recipient plant.

According to certain embodiments, the genetic element in isolated from the donor plant and introduced to the recipient plant by transformation or genome editing as is known in the art and as described hereinabove.

According to certain embodiments, examining the nucleic acid sample obtained from each offspring cultivated tomato plants for the presence of the at least one allele comprises identifying at least one nucleic acid marker indicative of said allele.

This method can be defined as “marker assisted selection” as the selection of the desired high-zeaxanthin phenotype is performed using nucleic acid markers specific for the high-zeaxanthin genotype. Since the high-zeaxanthin phenotype can only be properly identified phenotypically when the plant has produced fruit, it is of particular advantage that the establishment of proper introgression of the alleles in offspring plants may be monitored by using the gene specific markers.

Any method for obtaining a genetic material from the offspring tomato cultivar and any suitable molecular marker as are known in the art can be used for selecting plant comprising the high-zeaxanthin genotype according to the teachings of the present invention.

As used herein, the terms “molecular marker” or “molecular markers” refer to a molecular indicator that is used in methods for visualizing differences in characteristics of nucleic acid sequences. Examples of such indicators are diversity array technology (DArT) markers, restriction fragment length polymorphism (RFLP) markers, amplified fragment length polymorphism (AFLP) markers, single nucleotide polymorphisms (SNPs), sequence-characterized amplified regions (SCARs), cleaved amplified polymorphic sequence (CAPS) markers, sequence-characterized hybridization markers; or any combination thereof. According to certain exemplary embodiments, the step of examining a nucleic acid sample obtained from each offspring cultivated tomato plants for the presence of at least one allele of the invention comprises the use of a set of bi-directional primers. Bi-directional means that the orientation of the primers is such that one functions as the forward and one as the reverse primer in an amplification reaction of nucleic acid. The bi-directional primers are typically used in an amplification reaction on genomic DNA that amplifies a unique nucleic acid sequence indicative of the alleles of the invention.

According to certain embodiments, the pair of primers is designed to amplify an B^{sh} allele marker comprises a nucleic acid sequence at least 90% identical to the nucleic acid sequence set forth in SEQ ID NO:4. According to certain exemplary embodiments, the B^{sh} allele marker is amplified by a pair of primer comprising the nucleic acid sequence set forth in SEQ ID NO:2 and SEQ ID NO:3.

According to certain embodiments, the pair of primers is designed to amplify an hp3 allele marker comprising a nucleic acid sequence at least 90% identical to the nucleic acid sequence set forth in SEQ ID NO:12, wherein the nucleic acid comprises the nucleotide A at position 562. According to certain exemplary embodiments, the hp3 allele marker is amplified by a pair of primer comprising the nucleic acid sequence set forth in SEQ ID NO:10 and SEQ ID NO:11.

According to certain embodiments, the pair of primers is designed to amplify a gs allele marker comprising the nucleic acid sequence set forth in SEQ ID NO:[15]. According to certain exemplary embodiments, the hp3 allele marker is amplified by a pair of primer comprising the nucleic acid sequence set forth in SEQ ID NO:13 and SEQ ID NO:14.

According to certain embodiments, the pair of primers is designed to amplify an hp2^{d} allele marker comprising a nucleic acid sequence at least 90% identical to the nucleic acid sequence set forth in SEQ ID NO:18. According to certain exemplary embodiments, the hp3 allele marker is amplified by a pair of primer comprising the nucleic acid sequence set forth in SEQ ID NO:16 and SEQ ID NO:17.

Nevertheless, it is to be explicitly understood that these method aspects of the invention are not limited to the use of the markers identified herein, and that methods of the present invention may also make use of markers not explicitly disclosed herein or even yet to be identified, as identifying and using such markers is well within the skills of a person with knowledge in the Art.

In an additional or alternative method, the offspring cultivated tomato plants are examined for zeaxanthin content by biochemical analyses as are known in the art and as described in the Examples section hereinbelow.

According to certain aspects, the present invention provides a genetically modified tomato plant having fruit with high zeaxanthin content, the tomato plant comprising within its genome a combination of (i) at least one HIGH-BETA (B) allele; (ii) at least one HIGH-PIGMENT 3 (hp3) allele, and (iii) at least one green-stripe (gs) allele, wherein the zeaxanthin content in the tomato fruit is at least 20% out of the total carotenoid content.

According to certain embodiments, the genetically modified plant is transgenic plant.

The HIGH-BETA (B) allele; HIGH-PIGMENT 3 (hp3) allele, green-stripe (gs) allele are as described hereinabove.

According to another aspect, the present invention provides a transgenic tomato plant having fruit with high zeaxanthin content, the tomato plant comprising a combination of (i) a polynucleotide encoding beta-carotene hydroxylase (BCH); (ii) at least one HIGH-BETA (B) allele; and (ii) at least one HIGH-PIGMENT 3 (hp3) allele, wherein the zeaxanthin content in the tomato fruit is at least 20% out of the total carotenoid content.

According to certain embodiments, the BCH is a plant BCH. According to certain exemplary embodiments, the BCH is encoded by a polynucleotide at least 80% homologous to the Citrus clementina BCH2 gene (NCBI Accession number: XM_006421968). According to certain exemplary embodiments, the BCH is Citrus clementina BCH2. According to certain exemplary embodiment, the C. clementina BCH2 is amplified by a pair of primers, the pair of primers comprising the nucleic acid sequence set forth in SEQ ID NO:29 and the nucleic acid sequence set forth in SEQ ID NO:24.

According to certain embodiment, the C. clementina BCH2 comprises a marker amplified by a pair of primers, the pair of primers comprising the nucleic acid sequence set forth in SEQ ID NO:23 and the nucleic acid sequence set forth in SEQ ID NO:24.

According to certain embodiments, the BCH is a bacterial BCH encoded by a crtZ gene. According to certain embodiments, the bacterial crtZ gene is Pantoea ananatis crtZ accession No. D90087.

According to certain exemplary embodiments, the transgenic plant further comprises at least one allele selected from the group consisting of whiteflower (wf) allele, phytoene desaturase (crtI) allele and a combination thereof.

According to certain embodiments, the wf allele is amplified by a pair of primers, the pair of primers comprising the nucleic acid sequence set forth in SEQ ID NO:21 and the nucleic acid sequence set forth in SEQ ID NO:22.

According to certain embodiments, the CrtI allele is amplified by a pair of primers, the pair of primers comprising the nucleic acid sequence set forth in SEQ ID NO:25 and the nucleic acid sequence set forth in SEQ ID NO:26.

It is to be explicitly understood that the tomato plants of the invention, producing tomato fruit with zeaxanthin content of at least 20% of the total carotenoid content can be used in breeding programs to further elevate the zeaxanthin content within the fruit. As exemplified hereinbelow, crossing a high-zeaxanthin tomato plant of the genotype 30 hp3/B^{sh}/gs with high-zeaxanthin tomato plant of the genotype hp3/B^{sh}gs /hp2^{dg} resulted in zeaxanthin content of more than 50 μg/g Fruit FW.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Methods

Plant material and growth condition. Tomato (Solanum lycopersicum) cultivar M82 served as a reference “wild-type”. The tomato mutant HIGH-BETA (allele B^{sh}) plant was isolated by visual screening for orange fruit in a population of widespread heirloom tomato lines and was identified as Jaune Flamee of an unknown genetic background with a phenotype reshowing indeterminate growth and orange colored fruit. Mutants high-pigment 3 (hp3-1) (e1472) and white-flower 1 (wf1-2) (e1827) were previously described (Galpaz N et al., 2008. The Plant Journal, 53, 717-730; Galpaz N et al., 2006, ibid). Mutant green-stripe (gs), was originated from line LA0212 (Tomato Genetics Resource Center, Davis; tgrc.ucdavis.edu/Monogenic %20stock %201ist-2014.pdf) and was found in an heirloom tomato line of unknown background. Mutants green-flesh (gf) “Nyagous” cultivar and high-pigment 2 (hp2^{dg}) were obtained from the collection of Prof Zamir (The Hebrew University of Jerusalem, Israel). Plants were grown in the greenhouse for crossings as described (Neuman H et al., 2014. The Plant Journal, 78, 80-93). Phenotypic characterization was verified over four growing seasons in plants grown in greenhouses and in open fields. The transgenic 35S:CRTI (Romer S et al., 2000. Nat. Biotechnol., 18, 666-669) were kindly provided by Prof. Paul D. Fraser (Royal Holloway, University of London).

Pigment extraction and analysis. Fresh samples of fruit and leaves were collected from three biological replicates. Leaf pigments were extracted from 500 mg of young leaves, incubated with acetone over-night in Eppendorf tubes. The tissue was ground and, following centrifugation, the acetone phase was dried under stream of N₂. Fruit pigments were extracted from 150-250 mg of fresh pericarp tissue of fruit at the ‘mature-green’ or ‘ripe’ stages. The tissue was ground in 1 ml of 1:1 water chloroform mixture. The chloroform phase was separated by centrifugation and dried under a stream of N₂. The dried carotenoid extracts were dissolved in 500 μl acetone. Carotenoids were separated by high performance liquid chromatography (HPLC) using a Waters system consisting of a Waters 600 pump, Waters 996 photodiode array detector and Waters 717 plus Autosampler (Waters, Milford, MA). The static phase consisted of Spherisorb® ODS2 C18 reversed-phase column from Phenomenex (silica 5 m, 3.2 mm 250 mm) (Phenomenex®, Torrance, CA USA) and the mobile phase consisted of a solvent gradient as described in Table 1 at a constant flow of 1.6 ml/min (Table 1). The spectrum between 200 and 700 nm was recorded at a rate of one full spectrum per second. The carotenoids were identified according to their typical retention time certified by standards and characteristic absorption spectra. PGP-21

TABLE 1
The solvent gradient procedure used for separation of carotenoids
by HPLC at a constant flow of 1.6 ml/min.

| Time (minutes) | Acetonitrile:H₂O (9:1) | Ethyl acetate |
|---|---|---|
| 0-8 | 100% | 0% |
| 8-12 | 80% | 20% |
| 12-26 | 65% | 35% |
| 26-26.1 | 45% | 55% |
| 26.1-33 | 0 | 100% |

DNA extraction and genotyping using DNA markers. Young tomato leaves samples of approximately 15 mg were used for DNA extraction as described previously (Eshed, Y and Zamir D. 1995. Genetics, 141, 1147-1162). The genotyping of the homozygous mutants was confirmed by Cleaved Amplified Polymorphic Sequence (CAPS) or by sequencing. Primers and CAPS restriction enzymes for genotyping are described in Table 2.

RNA extraction and measurement of mRNA with Quantitative Real-Time RT-PCR. RNA extraction from fruit at the ‘ripe’ stage was performed from approximately 200 mg powder tissue with TRI Reagent RNA isolation reagent (Sigma-Aldrich) according to the manufacturer's protocol. Reverse transcription and DNase treatment was performed with iScript™ gDNA Clear cDNA Synthesis Kit #172-5035. To eliminate genomic DNA contamination, the cDNA was amplified using ACTIN primers (Table 2) that differentiate between genomic DNA and cDNA sequences. The cDNA was amplified by Applied Biosystems ProFlex PCR System Quantitative polymerase chain reactions were performed using the Applied Biosystems™ Fast SYBR™ Green Master Mix on a StepOnePlus™ Real-Time PCR System (Thermo Fisher Scientific, Eldan Electronic Instruments Ltd. Israel). Cycling conditions were 95° C. for 20 sec, followed by 40 cycles of 95° C. for 3 sec, 60° C. for 30 sec and fluorescence acquisition at 60° C. For each gene, the relative mRNA level was determined in three biological replicates. The gene ACTIN served as a control for normalization. Primers used for RT-PCR amplifications are described in Table 2.

Expression of BCH2 from C. clementina in E. coli. The cDNA of β-carotene hydroxylase (BCH2) from Citrus clementina was obtained from RNA isolated from pulp of fresh fruit followed by RT-PCR using primers 5′-AGCCACTAGTTGCCCGCGTGGCCGAGAAATTG-3′ (forward, SEQ ID NO:27) and 5′-GTCGCTCGAGCTGATCCAAAAATTGGTCCTC-3′ (reverse, SEQ ID NO:28). For expression in E. coli, 267 nucleotides were truncated from the 5′ end of the predicted mRNA (NCBI Reference Sequence: XM_006421968.1), which includes part of the presumed transit peptide. The remaining cDNA sequence from nucleotide 267 to 936 was cloned into the plasmid pBluescript SK+ between the SpeI and XhoI sites. The insert was sequenced to identify possible PCR-derived mutations. The plasmid was transfected to E. coli strain XL1-Blue grown on Luria-Bertani (LB) containing plasmid pAC-BETAipi, which carries Erwinia herbicola ipi, crtE, crtB, crtI, crtY was used (Cunningham F X Jr. and Gantt,E. 2007. Photosynth. Res., 92, 245-259). To enhance the expression of BCH2, 24 mg/L of Isopropyl 1-thio-β-D-galactopyranoside (IPTG) were added to the LB medium. E. coli cells were grown overnight at 37° C. on LB solid plates followed by 5 days at room temperature for pigment accumulation.

Constructs design and tomato transformation. Full-length cDNA of BCH2 from C. clementina fruit tissue was used as template for PCR amplification using the following primers: 5′-CCACAATCCACAATCCACTTC-3′ (Forward, SEQ ID NO:29) and 5′-TGATCCAAAAATTGGTCCTC-3′ (Reverse, SEQ ID NO:24) and cloned into the multiple cloning site of the plasmid pJET 1.2 (CloneJET PCR Cloning Kit #K1231, Thermo Fisher Scientific). The insert was sequenced to confirm its integrity and was excised from the plasmid by the restriction enzymes XhoI and XbaI. The insert was cloned into the XhoI and XbaI sites of the intermediate plasmid pART7, then cleaved by the restriction enzyme NotI and ligated into the T-DNA binary vector pART27 behind the CaMV constitutive promoter 35S, with Nos terminator and NPTII for kanamycin resistance (Gleave, A P. 1992. Plant Mol Biol., 20, 1203-1207). This plasmid was designated pBCH2-pART27. The plasmid pBCH2-pART27 was transfected into Agrobacterium tumefaciens (GV3101) by electrophoresis. Transformed Agrobacterium culture was incubated for 15 min with detached 10-day old tomato cotyledons of the homozygous double mutant hp3/B^{sh}, obtained in F₂ progenies of a cross between the high-pigment 3 (hp3) (Galpaz et al., 2008, ibid) and high-Beta B^{sh} (this work). Cotyledons were placed on feeder plates and after 48 hours transferred to SL1 medium (Jones medium with 400 mg/l carbenicillin, 1 mg/l zeatin, 100 mg/l kanamycin and 0.7% agar). After 14 days the cotyledons were transferred to SL2 medium (Jones medium with 250 mg/l carbenicillin, 100 mg/l Kanamycin, 1 mg/l zeatin, 0.5 mg/l zeatin riboside and 0.8% agar). Calli were excised from the cotyledons after a week or so and transferred again to SL2 medium plates. Shoots emerging from the calli were transferred to rooting medium (Nitsch medium with 150 mg/l carbenicillin, 50 mg/l Kanamycin, 50 mg/I Kanamycin and 1-2 mg/l IBA) for additional two weeks and then transferred to a transparent container with wet soil. Sturdy plantlets were finally transferred to 4-liter pots in the greenhouse. Transgenic plants were identified by PCR amplification the T-DNA sequence (primers described in Table 2 and Example 3).

TABLE 2
Primer used for genotyping of the different tomato mutants

| Mutant | | | | | | Restriction |
|---|---|---|---|---|---|---|
| name | symbol | Gene | Forward | Reverse | Type | enzyme |
| HIGH- | B^{Sh} | CycB | 5′-AGGGTTGTCAAAAATGTCTCA-3′ | 5′-AAAAGGTAATTTACTGAGTTGTGCAT- | length | |
| BETA | | | (SEQ ID NO: 2) | 3 | polymorphism | |
| | | | | (SEQ ID NO: 3)′ | | |
| HIGH- | hp3 | ZEP | 5′-AACCCACAAATCCCACTTTC-3′ | 5′-TTCTCTTCGGACAAGCACAC-3′ | CAPS | DraI |
| PIGMENT | | | (SEQ ID NO: 10) | (SEQ ID NO: 11) | | |
| 3 | | | | | | |
| GREEN | gf5 | SGR1 | 5′-CTCGATTTCAATTTCCTTCAGC-3′ | 5′-CCATCCTAAACTTGATGTTCTTGTC-3′ | length | |
| FLESH | | | (SEQ ID NO: 19) | (SEQ ID NO: 20) | polymorphism | |
| | | | | | (1164 bp | |
| | | | | | deletion) | |
| white- | wf | CrtR-b2 | 5′-ATCTTGTGGCAGCTGTGATG-3′ | 5′-TGACCTCCAACTTTCATAATGC-3′ | sequencing | |
| flower | | | (SEQ ID NO: 21) | (SEQ ID NO: 22) | | |
| C. | C. BCH2 | BCH2 | 5′-CTATCCTTCGCAAGACCCTTCC-3′ | 5′-TGATCCAAAAATTGGTCCTC-3′ | dominant | |
| clementina | | | (35S) | (SEQ ID NO: 24) | (transgenic) | |
| β-carotene | | | (SEQ ID NO: 29) | | | |
| hydroxylase | | | | | | |
| CrtI | CrtI | CrtI | 5′-CTATCCTTCGCAAGACCCTTCC-3′ | 5′-GCAAAACTTTTCGAGCCAAC-3′ | dominant | |
| | | | (35S) | (SEQ ID NO: 26) | (transgenic) | |
| | | | (SEQ ID NO: 25) | | | |
| HIGH- | hp2dg | DET1 | 5′-TTCTTCGGATTGTCCATGGT-3′ | 5′- CACCAATGCTATGTGCCAAA-3′ | CAPS | AclI |
| PIGMENT | | | (SEQ ID NO: 16) | (SEQ ID NO: 17) | | |
| 2 | | | | | | |

### Example 1: Genetic Breeding of High zeaxanthin Tomato

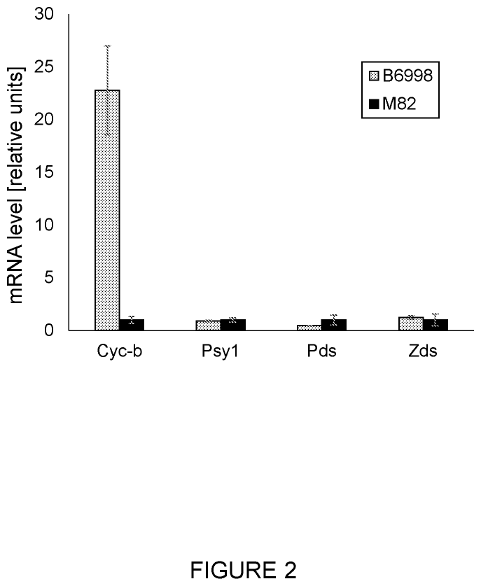

Tomato fruit accumulate mainly lycopene as an end carotenoid product due to down-regulation of expression of the lycopene cyclase genes Lcy-e encoding ε-cyclase and Lcy-b encoding the β-cyclase (Ronen et al., 1999, ibid; Pecker et al., 1996. Plant Mol. Biol. 30, 807-819; Hirschberg, 2001. Curr. Opin. Plant Biol. 4, 210-218). The first step toward obtaining beta-xanthophylls in tomato fruit is enhancing β-carotene synthesis. It was previously established that a chromoplast-specific lycopene β-cyclase, CycB, is responsible for β-carotene synthesis in tomato fruit (Ronen et al., 2000, ibid). Several dominant alleles of HIGH-BETA were isolated following introgression of the CycB (B) gene from wild tomato species (Tomes M L et al., 1956. Botanical Gazzete, 117, 248-253). An allele of HIGH-BETA was characterized in an heirloom tomato line known as Jaune Flamee of an unknown genetic background with a phenotype showing indeterminate growth and orange colored fruit. The genomic DNA sequence of CycB from this line (SEQ ID NO:1) is identical to the gene from the green-fruited wild species S. habrochaites (LA0316) (Dalal et al., 2010. ibid) (The C. M. Rick Tomato Genetics Resource Center, TGRC, tgrc.ucdavis.edu/index.aspx), which is also found in the heirloom variety Jaune Flamme, NCBI Accession KP233161 (Orchard, C J. 2014. Naturally occurring variation in the promoter of the chromoplast-specific Cyc-B gene in tomato can be used to modulate levels of α-carotene in ripe tomato fruit. 1-90. Ph.D. Thesis, Graduate Program in Horticulture and Crop Science, The Ohio State University, 2014). Therefore, this allele was termed B^{sh}. Fruits of B^{sh} accumulated β-carotene up to 80% of total carotenoids (Table 3). The intermediate concentration of β-carotene in fruit of F1 hybrid of B^{sh}×M82 compared with parental line B^{sh}, fits the semi-dominance nature of the allele (Table 3). Expression of CycB in B^{sh} lines during ripening, measured by qRT-PCR, was 40-fold higher than in the wild type variety M82, while expression of other genes in the carotenoid biosynthesis pathway, such as phytoene desaturase (PDS), zeta-carotene desaturase (ZDS), and phytoene synthase 1 (PSY1), did not significantly change (FIG. 2).

TABLE 3
Carotenoid concentration in ripe fruits (μg/g fresh weight, FW).

| | Phytoene + | | β- | β- | | | | Total | Percent |
|---|---|---|---|---|---|---|---|---|---|
| Line/Genotype | Phytofluene | Lycopene | carotene | cryptoxanthin | Zeaxanthin | Lutein | Others | carotenoids | β-xanth. |
| M82 | 8.8 ± 2.8 | 45.8 ± 7.3 | 1.0 ± 0.2 | | 0.1 ± 0.1 | 0.4 ± 0.2 | 0.8 | 56.9 ± 10.1 | <1 |
| B^{Sh a} | 0.9 ± 0.4 | 2.2 ± 0.7 | 22.4 ± 1.8 | 0.1 ± 0.1 | | 0.4 ± 0.2 | 1.4 | 27.3 ± 2.0 | <1 |
| B^{Sh}x M82 (F1) | 3.0 ± 0.8 | 16.7 ± 2.5 | 15.4 ± 2.0 | 0.1 | | 0.7 ± 0.1 | 2.5^{b} | 38.3 ± 4.4 | <1 |
| hp3 | 6.3 ± 1.2 | 26.6 ± 6.7 | 3.5 ± 0.4 | | 5.2 ± 0.3 | 2.7 ± 0.9 | 0.5 | 44.7 ± 7.6 | 11.6 |
| Green-stripe (gs) | 7.6 ± 1.2 | 78.6 ± 13.8 | 11.8 ± 0.8 | | | 1.0 ± 0.1 | | 99.1 ± 13.7 | |
| Green-flesh (gf) | 4.2 ± 1.4 | 25.6 ± 8.4 | 2.9 ± 0.7 | | | 2.1 ± 0.2 | 1.7 | 33.9 ± 9.3 | 0 |
| hp3\|B^{Sh} | 1.0 ± 0.3 | 1.6 ± 0.8 | 26.1 ± 3.5 | 0.3 ± 0.1 | 7.7 ± 0.6 | 1.1 ± 0.6 | 0.3 | 38.5 ± 4.4 | 20.8 |
| hp3\|gf | 8.5 ± 1.5 | 81.3 ± 7.2 | 7.9 ± 1.3 | | 7.8 ± 0.5 | 3.1 ± 0.3 | 0.2 | 108.8 ± 8.3 | 7.2 |
| hp3\|gs | 9.6 ± 0.4 | 34 ± 1.0 | 2.2 ± 0.3 | | 4.2 ± 0.2 | 0.6 ± 0.1 | | 50.6 ± 3.8 | 8.3 |
| hp3/B^{Sh}/gf | 3.3 ± 0.8 | 1.8 ± 1.2 | 33.8 ± 6.0 | 0.5 ± 0.2 | 7.0 ± 1.4 | 0.7 ± 0.1 | 4.5^{c} | 53.3 ± 7.6 | 14.1 |
| hp3/B^{Sh}/gs | | | 24.3 ± 3.7 | 1.0 ± 0.1 | 16.3 ± 2 | 0.6 ± 0.3 | | 42.6 ± 3.9 | 40.6 |
| hp3/B^{Sh}/gs/hp2^{dg} | 1.0 ± 0.1 | 0.1 | 26.7 ± 0.3 | 0.6 | 34.8 ± 0.8 | 1.5 ± 0.2 | 0.5 | 65.5 ± 1.4 | 53.7 |
| #1 | | | | | | | | | |
| hp3/B^{Sh}/gs/hp2^{dg} | 2.2 ± 0.3 | 0.8 | 37.2 ± 2.2 | 1.1 ± 0.1 | 32.4 ± 2.4 | 0.9 ± 0.1 | 0.8 | 75.7 ± 4.2 | 44.4 |
| #2 | | | | | | | | | |
| hp3/B^{Sh}/gs/hp2^{dg} | 1.8 ± 0.5 | | 34.0 ± 2.2 | 0.8 | 25.4 ± 2.2 | 1.4 ± 0.5 | 1.2 | 64.5 ± 4.5 | 40.6 |
| #3 | | | | | | | | | |
| hp3/B^{Sh}/gs/hp2^{dg} | 0.8 ± 0.1 | | 21.4 ± 2.4 | 0.6 | 22.6 ± 0.8 | 0.6 ± 0.3 | 1.6 | 47.5 ± 3.8 | 48.8 |
| #4 | | | | | | | | | |
| hp2^{dg} | 29.0 ± 2.4 | 105.1 ± 4.9 | 9.4 ± 0.3 | | | | | 148.1 ± 4.1 | |

^{a}Jaune Flamme line of unknown genetic background; ^{b}γ-carotene 2.2; ^{c}γ-carotene 3.7

Mutant B^{sh} was crossed with the M82 mutant HIGH-PIGMENT 3 (hp3), in which the gene for zeaxanthin epoxidase (ZEP) is impaired (Galpaz et al., 2008, ibid). Leaves of hp3 lack violaxanthin and contain 85% less of neoxanthin compare to the wild type M82, due to obstruction of zeaxanthin conversion to downstream xanthophylls (Galpaz et al., 2008, ibid and Table 4). Both leaves and fruit of hp3 contain significantly higher amounts of zeaxanthin (Tables 3 and 4). A double homozygous mutant B^{sh} hp3 was identified using unique CAPS markers in F2 offspring of a cross between the mutants. Accumulation of zeaxanthin in fruit of the double mutant B^{sh} hp3 was 50% higher than in the hp3 single mutant (Table 3). Even though the conversion of lycopene to β-carotene was efficient in B^{sh}/hp3, only small fraction of it was utilized for the synthesis of beta-xanthophylls.

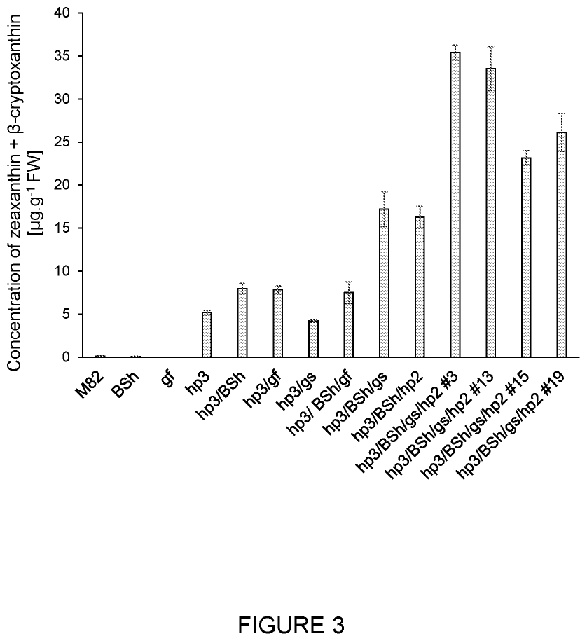

To increase xanthophyll synthesis, the double mutant B^{sh}/hp3 was crossed with two different recessive mutants: The GREEN FLESH mutant STAY-GREEN, which is impaired in the SlSGR1 protein (Luo Z et al., 2013. New Phytol., 198, 442-452) and green-stipe (gs) of a gene recently shown to be a methylated isoform of TAGL1, regulating diversified chloroplast development and carotenoid accumulation (Liu G et al., 2020. New Phytol. doi: 10.1111/nph.16705). Fruit of various green-flesh tomato varieties contain higher concentration of total carotenoids, including xanthophylls of the photosynthetic apparatus (Luo et al., 2013, ibid). The concentration of zeaxanthin in fruit of the triple mutant B^{sh}/hp3/gs increased more than two folds compared to the double mutant B^{Sh}/hp3, to a significant level of 16 μg/g FW, and also contained 1 μg/g FW of 3-cryptoxanthin (Table 3, FIG. 3).

The triple mutant B^{sh}/hp3 gs line was crossed with the HIGH-PIGMENT mutant hp2^{dg} (Levin I et al., 2006, ibid) to generate a quadruple homozygous mutant B^{sh}/hp3/gs/hp2^{dg}, which was screened and verified by genotyping of F2 plants. The recessive mutation hp2 in the DE-ETIOLATED 1 (DET1) gene (Mustilli et al., 1999, ibid) increases plastid compartment size in leaf and fruit cells thus elevates chlorophyll levels in immature fruit and total carotenoids in ripe fruit (Azari R et al., 2010. Biotechnology Advances, 28, 108-118). Carotenoid composition of the four lines from this population exhibited the highest level of zeaxanthin accumulation (Table 3, FIG. 3).

TABLE 4
Carotenoid composition in tomato leaves (μg/g FW)

| | | | | | | | Total |
|---|---|---|---|---|---|---|---|
| line | β-carotene | Zeaxanthin | Antheraxanthin | Violaxanthin | Neoxanthin | Lutein | carotenoids |
| M82 | 6.1 ± 0.4 | 0.8 ± 0.2 | 1.1 ± 0.1 | 22.7 ± 2.6 | 32.7 ± 2.3 | 82.1 ± 6.3 | 145.5 ± 11 |
| CrtI | 5 ± 1.4 | 1.6 | 3.6 ± 0.4 | 24.2 ± 3.1 | 19.8 ± 1.0 | 38.9 ± 0.6 | 93.1 ± 3.9 |
| hp3 | 2.4 ± 0.3 | 27.3 ± 3.6 | 4.1 ± 0.9 | | 3.7 ± 0.9 | 51.7 ± 14.2 | 89.2 ± 13.8 |
| hp3/B^{Sh}/wf/ | 2.1 ± 0.9 | 26.5 ± 4.8 | 12.1 ± 3 | 2.7 ± 0.9 | 16.7 ± 2.8 | 22 ± 2.3 | 82.2 ± 13.3 |
| CcBCH2#1 | | | | | | | |

### Example 2: Further Breeding of the high-zeaxanthin Tomato Plants

line hp3/B^{sh}gs/ hp2^{dg}#1 (Table 3) was self-pollinated to form F3 offspring. The zeaxanthin in the F3 plants reached 39.0 μg/g FW, which equal to 557 μg/g in dry weight. This line, which also contained 37 μg/g FW (529 μg/g dry weight (DW)) β-carotene, was named “Xantomato”.

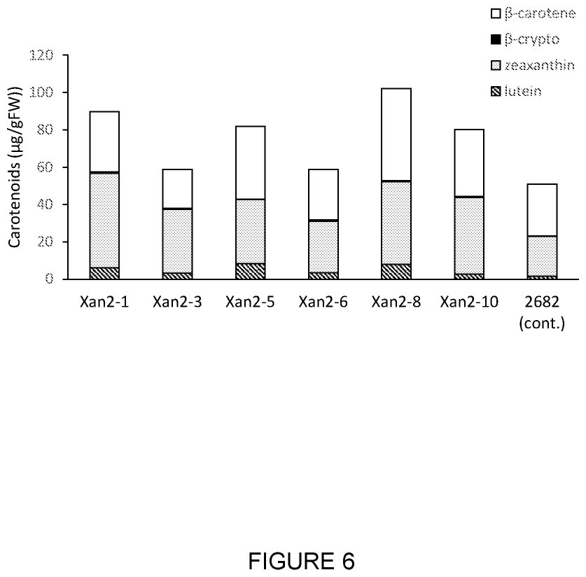

The breeding lines of Xantomato were composed of two different populations that showed phenotypic variability in fruit carotenoid composition. One was homozygous in the high-pigment 2 allele hp2^{dg} and the second did not contain this mutation. Nevertheless, the fruit of the latter population contained a high concentration of zeaxanthin. A cross between plants of Xantomato (first population, hp3/B^{sh}/gs hp2^{dg}) with high-zeaxanthin lines from the other population that did not carry hp2^{dg} (hp3/B^{sh} gs) yielded F2 plants homozygous for the hp2^{dg} allele with fruit containing zeaxanthin at a concentration of 50.7±4.2 μg/gr fresh weight (FIG. 6).

### Example 3: Transgenic Breeding of high zeaxanthin Tomato

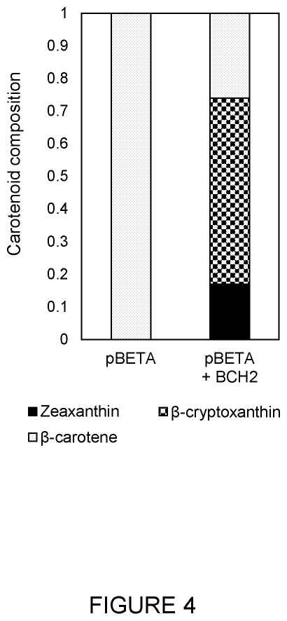

An alternative approach to enhancing the hydroxylation of β-carotene in tomato fruit is by transgenic expression of β-carotene hydroxylase (BCH2). Considering the high levels of β-xanthophylls that accumulate in Citrus, the BCH2 gene from Citrus clementina was selected. Fruits of this species also accumulate significant amount of β-cryptoxanthin (Dhuique-Mayer C et al., 2005. Journal of agricultural food chemistry, 53, 2140-2145). The full-length cDNA of BCH2 from Citrus clementina (NCBI Reference Sequence: XM_006421968.1) was amplified from mRNA isolated from C. clementina fruit through reverse transcription and polymerase chain reaction (RT-PCR), and its identity was confirmed by sequencing. The cDNA of C. clementina, without the first 89 codons that encode the predicted transit peptide, was cloned into the plasmid vector pBCH2 for high expression in Escherichia coli to obtain the recombinant plasmid pBCH2. Plasmid pBCH2 was transfected into a β-carotene-producing E. coli that carries plasmid pBETA (Cunningham and Gantt, 2007, ibid). Carotenoid analysis showed that the C. clementina BCH2 hydroxylated β-carotene in E. coli (FIG. 4). The level of zeaxanthin in E. coli cells was lower than that of β-cryptoxanthin, suggesting that β-carotene di-hydroxylation by C. clementina BCH2 in E. coli cells is less efficient than the mono-hydroxylation (FIG. 4).

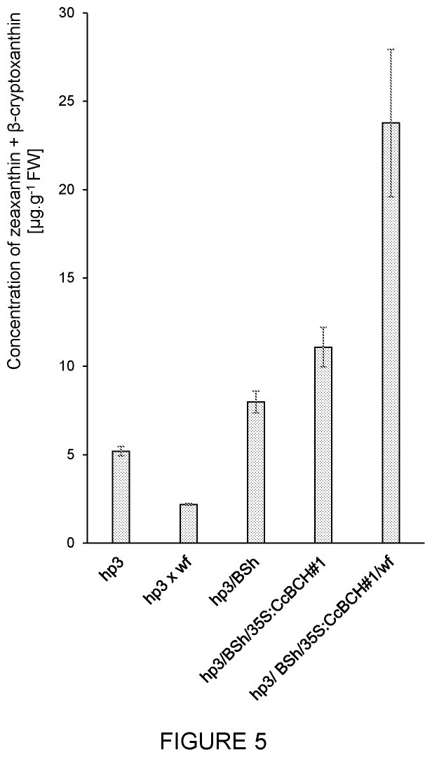

Next, the C. clementina BCH2 full-length cDNA was cloned behind the constitutive promoter CaMV 35S in the binary vector pBCH2-pART27. Plasmid pBCH2-pART27 was transferred to the hp3/B^{sh} double-mutant tomato plant using Agrobacterium transformation. Seven independent transgenic plants of hp3/B^{sh} 35S:CcBCH2 were obtained. Some of the transgenic plants showed yellower fruit at the mature green stages compared to the corresponding parent hp3/B^{sh}. Carotenoid analysis indicated that the color change is due to significant accumulation of zeaxanthin in the mature-green fruit (Table 4). Carotenoid composition in fruit of T1 plants revealed a wide range of xanthophyll accumulation in different transgenic lines (Table 4). Interestingly, in most of the lines the amount of xanthophylls was lower than in the parental line hp3/B^{sh}. This phenotype could arise from co-suppression of the endogenous tomato β-carotene hydroxylase by the transgenic C. clementina BCH2. Most of the β-xanthophylls accumulated as zeaxanthin with only small amount of β-cryptoxanthin (Table 4). The transgenic line that accumulates the highest levels of xanthophyll, hp3/B^{sh}-C. BCH2-1, (FIG. 5) was chosen for further breeding in this project.

To eliminate possible negative effects of co-suppression by the endogenous β-carotene hydroxylase, the line hp3/B^{sh}-CcBCH2-1 was crossed with the mutant white-flower (wf), which carries a mutation in the chromoplast-specific β-carotene hydroxylase BCH2 (CrtR-b2) (Galpaz et al., 2006). Fruit of the homozygous triple mutant hp3/B^{sh/}wf contained significantly higher concentration of zeaxanthin (Table 4). Addition of a transgenic BCH2-1 expression to the triple mutant in the line hp3/B^{sh}wf/CcBCH2#1, increased xanthophyll accumulation in the ripe fruit to a level of 23.8 μg/g FW. The xanthophyll level of this mutant was nearly 5-fold higher than in the corresponding parent, hp3, and was not accompanied by accumulation of β-carotene, which is probably the reason for reduction in total fruit carotenoids (Table 4 and FIG. 5).

The triple mutant hp3/B^{sh}/CcBCH2#1 was crossed with a tomato transgenic line that over-expressed the bacterial phytoene desaturase gene, crtI, under control of constitutive CaMV 35S promoter (Romer et al., 2000). The xanthophylls in hp3/B^{sh}/ CcBCH2#1/CrtI fruit increased, with a significant elevation of the β-carotene levels compares to hp3/B^{sh}/CcBCH2#1 (Table 5, FIG. 5).

TABLE 5
Carotenoid accumulation (μg/g FW) of transgenic tomato ripe fruit

| | Phytoene + | | β- | β- | | | | Total |
|---|---|---|---|---|---|---|---|---|
| line | Phytofluene | Lycopene | carotene | cryptoxanthin | Zeaxanthin | Lutein | Others | carotenoids |
| Wf^{a} | | 35 ± 5.2 | 1.7 ± 1.5 | | | 0.7 ± 0.1 | | 42.2 |
| hp3\|wf | 14.0 ± 1.1 | 295.2 | | | 2.2 ± 0.1 | 2.1 ± 0.1 | | 53.8 ± 3.8 |
| hp3\|B^{Sh}/CcBCH2#1 | 0.1 ± 0.1 | 0.1 ± 0.1 | 2.0 ± 0.4 | 0.5 ± 0.1 | 10.6 ± 1.1 | 0.5 ± 0.1 | | 14.1 ± 1.6 |
| hp3\|B^{Sh}/CcBCH2#3 | 3.5 ± 0.8 | 3.2 ± 0.6 | 24.7 ± 3.2 | | 2.0 ± 0.3 | 0.3 ± 0.1 | 0.9 | 36.1 ± 3.8 |
| hp3\|B^{Sh}/CcBCH2#4 | 5.0 ± 2.5 | 3.6 | 48.9 ± 6.2 | | 2.2 | 2.1 | 1.6 ^{b} | 57.3 |
| hp3\|B^{Sh}/CcBCH2#5 | | | 0.41 ± 0.1 | 0.2 | 3.6 ± 0.6 | | | 4.2 ± 0.8 |
| hp3\|B^{Sh}/CcBCH2#6 | | | 7.8 ± 0.8 | 0.5 ± 0.1 | 6.2 ± 0.9 | 0.1 ± 0.1 | | 14.6 ± 1.6 |
| hp3\|B^{Sh}/CcBCH2#7 | | | 2.1 ± 0.8 | 0.3 ± 0.1 | 4.2 ± 1.0 | | | 7.5 ± 1.6 |
| hp3\|B^{Sh}/CcBCH2#9 | | | 26.1 ± 9.1 | | 3.1 ± 0.4 | 0.7 ± 0.2 | 0.9 | 32.1 ± 12 |
| hp3\|B^{Sh}/CcBCH2#1/CrtI | | | 26.4 ± 7.2 | 0.6 ± 0.1 | 16.3 ± 1.5 | 1.9 ± 0.4 | 2.2 | 47.4 ± 8.5 |
| hp3\|B^{Sh}/wf | 3 ± 0.3 | | 49.2 ± 4.8 | 0.4 ± 0.4 | 13.6 ± 0.2 | 1.3 ± 0.1 | | 67.5 ± 4.9 |
| hp3\|B^{Sh}/wf/ CcBCH2#1 | | | 2.6 ± 0.3 | 1.2 ± 0.3 | 22.6 ± 3.9 | 1.5 ± 0.3 | | 28.9 ± 4.2 |

^{a}from Galpaz et al., 2006; ^{m}ainly γ-carotene;

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A tomato plant having fruit with high zeaxanthin content, the tomato plant comprising within its genome a combination of (i) at least one HIGH-BETA (B) allele; (ii) at least one HIGH-PIGMENT 3 (hp3) allele, and (iii) at least one green-stripe (gs) allele, wherein the zeaxanthin content in the tomato fruit is at least 20% out of the total carotenoid content.

2. (canceled)

3. The tomato plant of claim 1, wherein the HIGH-BETA allele encoding a lycopene β-cyclase is derived from Solanum habrochaites (B^{sh} allele); the hp3 allele encodes a mutant zeaxanthin epoxidase (ZEP) having a reduced or no capability to convert zeaxanthin to violaxanthin; and the gs allele comprises a polynucleotide marker amplified by a pair of primers comprising a pair of oligonucleotides having a nucleic acid sequence selected from the group consisting of SEQ ID NO:13 and SEQ ID NO:14.

4-5. (canceled)

6. The tomato plant of claim 3, wherein the HIGH-BETA allele comprises at least one polynucleotide marker selected from the group consisting of (i) a marker amplified by a primer pair comprising a pair of oligonucleotides having a nucleic acid sequence selected from the group consisting of SEQ ID NO:2 and SEQ ID NO:3: (ii) a marker amplified by a primer pair comprising a pair of oligonucleotides having a nucleic acid sequence selected from the group consisting of SEQ ID NO:5 and SEQ ID NO:6.

7-11. (canceled)

12. The tomato plant of claim 3, wherein the hp3 allele comprises a polynucleotide marker amplified by a primer pair comprising a pair of oligonucleotides having a nucleic acid sequence selected from the group consisting of SEQ ID NO:10 and SEQ ID NO:11.

13-14. (canceled)

15. The tomato plant of claim 1, wherein the green-stripe (gs) allele is an allele originated from tomato line LA0212.

16. The tomato plant of claim 1, wherein the tomato plant further comprises within its genome at least one HIGH-PIGMENT 2 mutant allele (hp2).

17. The tomato plant of claim 16, wherein the HIGH-PIGMENT 2 mutant allele is hp2^{dg} allele, the hp2^{dg} allele comprising a polynucleotide marker amplified by a primer pair comprising a pair of oligonucleotides having the nucleic acid sequence selected from the group consisting of SEQ ID NO:16 and SEQ ID NO:17.

18-19. (canceled)

20. The tomato plant of claim 1, wherein the zeaxanthin content in the tomato fruit is at least 25 μg/g fruit fresh weight (FW).

21. The tomato plant of claim 1, said plant is a transgenic plant.

22. A seed of the tomato plant of claim 1, wherein a plant grown from the seed comprises within its genome a combination of (i) at least one HIGH-BETA (B) allele; (ii) at least one HIGH-PIGMENT 3 (hp3) allele, and (iii) at least one green-stripe (gs) allele, and wherein the zeaxanthin content in a fruit of the plant is at least 20% out of the total carotenoid content in the fruit.

23. An isolated cell or a tissue culture obtained from the tomato plant of claim 1, wherein a plant regenerated from the isolated cell or tissue culture comprises within its genome a combination of (i) at least one HIGH-BETA (B) allele; (ii) at least one HIGH-PIGMENT 3 (hp3) allele, and (iii) at least one green-stripe (gs) allele, and wherein the zeaxanthin content in a fruit of the plant is at least 20% out of the total carotenoid content in the fruit.

24. A tomato fruit having zeaxanthin content of at least 20% out of the total carotenoid content in the fruit.

25. (canceled)

26. A method for elevating the content of zeaxanthin in tomato fruit relative to the total carotenoid content in the fruit, comprising expressing within the genome of a tomato plant producing said fruit at least one of (i) at least one HIGH-BETA (B) allele; (ii) at least one HIGH-PIGMENT 3 (hp3) allele, and (iii) at least one green-stripe (gs) allele.

27. The method of claim 26, wherein the tomato plant is selected from the group consisting of: (i) a plant devoid of the B allele, the hp3 allele, and the gs allele, thereby said method comprises expressing within the genome of said plant a combination of the three alleles; a plant comprises one allele selected from the group consisting of the B allele, the hp3 allele, and the gs allele, thereby said method comprises expressing within the genome of said plant the other two allele; and a plant comprises two alleles selected from the group consisting of the B allele, the hp3 allele, and the gs allele, thereby said method comprises expressing within the genome of said plant the other third allele.

28-30. (canceled)

31. The method of claim 26, wherein the HIGH-BETA allele is B^{sh}, the hp3 allele encodes a mutant zeaxanthin epoxidase (ZEP) having a reduced or no capability to convert zeaxanthin to violaxanthin; and the gs allele comprises a polynucleotide marker amplified by a pair of primers comprising a pair of oligonucleotides having a nucleic acid sequence selected from the group consisting of SEQ ID NO:13 and SEQ ID NO:14.

32-49. (canceled)

50. The method of claim 26, wherein said method comprising the steps of:
a. introducing into a recipient tomato plant at least one genetic element from at least one donor plant, the genetic element is selected from the group consisting of a genetic element comprising B allele; a genetic element comprising hp3 allele; and a genetic element comprising gs allele; thereby producing offspring tomato plants;
b. examining a nucleic acid sample obtained from each offspring tomato plants for the presence of the at least one allele;
c. selecting tomato plants comprising a combination of said B allele, hp3 allele and gs allele, thereby producing tomato plants having fruit with zeaxanthin content of at least 20% out of the total carotenoid content in the fruit.

51. The method of claim 50, said method comprises introducing a combination of genetic elements comprising the B allele, hp3 allele and gs allele.

52. The method of claim 50, wherein at least one exists: (i) each of the genetic elements is originated from a different donor plant: (ii) a combination of genetic elements is originated from a single donor plant.

53. (canceled)

54. A transgenic tomato plant having fruit with high zeaxanthin content, the tomato plant comprising a combination of (i) a polynucleotide encoding beta-carotene hydroxylase (BCH); (ii) at least one HIGH-BETA (B) allele; and (ii) at least one HIGH-PIGMENT 3 (hp3) allele, wherein the zeaxanthin content in the tomato fruit is at least 20% out of the total carotenoid content.

55. The transgrenic plant of claim 54, wherein the BCH is clementina BCH2.


SEQUENCE LISTING (29 sequences)

SEQ ID NO: 1 - DNA, 3949 bp, Solanum lycopersicum
tcagctgaagtttttaactagacattaaacatacttggcaaagcaatatgctaatcattattttattttgattttggctatcaacaaaagttttcctcattcagaaaacattagatgaatatgttattgttcaggaattggaaatgttgaaatttagtactccattagaaaagtagttagtaaatttgatgagaaacaacaatcaaaaaataaatttaaaaaattgactagaaacgtgaaactgcacctttatagattgaacttgaacatgattgacacagtatttatggttaaaatcctcaagttaattgaaaaaaagtcaattcgtttgtacgtaaaatttctgtccgtggttgaagttacttttttcttgtattacgtgttcattttttactttaagaacgtgcttttattgggtttttggagttagtaatcttccccagctgctattgtggtgattatcataaaacgattgcagtgggtagtagctagtcagtgctatttaggctcccccatttctagtcatttgatataaacaggtgcacaacaatggaaaaaaactatgtctcaatcccaaactagtcgatgtggtggactactttgcctacatccatccatgaggctggaaccgttgctcttcatatccagacaatattatgataatcccttatccatttttcatcatactccagtagcagtataacatgttcctaggttgggtgagcttaataaggttcaagatctcattgtatagcttgtcttttgtttcagtcgtcttaggcttgggttagttggtgttgttgtttcatacttctatcaaatatgactgttggaggaagtaatttacctttagttcgactagtctaatactctaactacatcaagatttgcatcattctcgtccaagaaagttcttagtttgaagccttttggtcttatatatttgtcaatctgagcttcgcaactttctcatgacaggggtttgttgacatgcctgattgtgctcttcctttacttgataattgctgcttgttgcggaggcatctttctaccttcctgcagatcatgaattctctgaaaaggagcaccatatttgccacactgtggttcatatttccaattacatttagatgaactatatcatcagagtgaaaggttattgtataatcaatccagtggattctcgttctggcacctttagaagtacatgtgcggaaaagaatgataaggtttgtattgttgttgacaaggcctgttgcctttctcatttgtaaatgttctgaacgactcctaaattactcttaaagtgtaaggtcttccgtgcctgtttgtaaatataatgctgtgccgcgacttaccttttgtaccatttgttcaaatgtatggcctggacactagggttgtcaaaaatgtctcatgacttcacccttctttcttgtcttggtgcccgttttattggtctgacaacggcgtgatgccaaatttctgccgctccacagtgagcatttcgatctactggaaattgaccaacttattttatcacttgataactagagtctgggttcaaacaaaatccaataactttaatcatacattgtatttatattgaaaaaaaattatgcacaactcagtaaattaccttttttagcagtcaaaaattctagatcagtttctaattaatcaaaatggcctttatagggtcccagttccactaatatacctgccgtccatgcactgactacaagacaaatacctcactatgtttgttagtgcttggtaatataaaaccttttcttttatgagaaagttcaccgaaaataattttctatttgtggcataactagtatcgaagtatatagtgcagattgacaagaatttaattttgcagttgggcacatgaacaattttcctcaaagttgtagaaaatatttttcattttcttgtcaccgaaaattatttataattgaaattgaaaccgaatgagctgcaagactcgagtcgaatttcaaaaaaattgaccaactaaatatgaaaaaattcgaaaatatcccccacccctgccaaacacatcctaaagtgaggtatagactgggactggcattgggaaaagggtaaaatgctttcattagcttagcaaagattccactttgttagctatctttctttctcatttcctcttttctttttcttttttttgttatataagccaaagtaggtacccaaaagcatcaatattttgtattgcttggtgattcctctttagtccagtatttcattttctgcaagtttcacctccctccataattaaccattatcaatctttacattttctataatggaaactcttctcaagccttttccatctcttttactttcctctcctacaccctataaatctattttccaacaaaatccctcttttctaagtcccaccaccaaaaaaaaatcaagaaaatgtcttcttagaaacaaaagtagtaaacttttttgtagctttcttgatttagcacccacatcaaagccagagtctttagatgttaacatctcatgggttgatcctaattcgaatcgggctcaattcgacgtgatcattatcggagctggccctgctgggctcaggctagctgaacaagtttctaaatatggtattaaggtatgttgtgttgacccttcaccactttccatgtggccaaataattatggtgtttgggttgatgagtttgagaatttaggactggaagattgtttagatcataaatggcctatgacttgtgtgcatataaatgataacaagactaagtatttgggaagaccatatggtagagttagtagaaagaagctgaagttgaaattgttgaacagttgtgttgagaacagagtgaagttttataaagctaaggtttggaaagtggaacatgaagaatttgagtcttcaattgtttgtgatgatggtaagaagataagaggtagtttggttgtggatgcaagtggttttgctagtgattttatagagtatgacaagccaagaaaccatggttatcaaattgctcatggggttttagtagaagttgataatcatccatttgatttggataaaatggtgcttatggattggagggattctcatttaggtaatgagccatatttaagggtgaataatgctaaagaaccaacattcttgtatgcaatgccatttgatagtaatttggttttcttggaagagacttctttggtgagtcgtcctgtgttatcgtatatggaagtgaaaagaaggatggtggcaagattaaggcatttggggatcaaagtgaaaagtgttattgaggaagagaaatgtttgatccctatgggaggaccacttccgcggattcctcaaaatgttatggctattggtgggaattcagggatagttcatccatcaacagggtacatggtggctaggagcatggctttagcaccagtactagctgaagccatcgtcgaggggcttggctcaacaagaatgataagagggtctcaactttaccatagagtttggaatggtttgtggcctttggatagaagatgtgttagagaatgttattcatttgggatggagacattgttgaagcttgatttgaaagggactaggagattgtttgacgctttctttgatcttgatcctaaatactggcaagggttcctttcttcaagattgtctgtcaaagaacttggtttactcagcttgtgtcttttcggacatggctcaaatttgactaggttggatattgttacaaaatgtcctgttcctttggttagactgattggcaatctagcaatagagagcctttgaatgtgaaaagtttgaatcattttctttattttcatttctttgattattttcatattttctcaatgcaaaagtgagagaagagcaatacactgtcaacaaataaactactatttggaa

SEQ ID NO: 2 - DNA, 21 bp, Artificial Sequence
  Synthetic Oligonucleotide
agggttgtcaaaaatgtctca

SEQ ID NO: 3 - DNA, 26 bp, Artificial Sequence
  Synthetic Oligonucleotide
aaaaggtaatttactgagttgtgcat

SEQ ID NO: 4 - DNA, 269 bp, Artificial Sequence
  Synthetic Polynucleotide
caaatgtatggcctggacactagggttgtcaaaaatgtctcatgacttcacccttctttcttgtcttggtgcccgttttattggtctgacaacggcgtgatgccaaatttctgccgctccacagtgagcatttcgatctactggaaattgaccaacttattttatcacttgataactagagtctgggttcaaacaaaatccaataactttaatcatacattgtatttatattgaaaaaaaattatgcacaactcagtaaattacctttt

SEQ ID NO: 5 - DNA, 20 bp, Artificial Sequence
  Synthetic Oligonucleotide
ccatgcactgactacaagac

SEQ ID NO: 6 - DNA, 20 bp, Artificial Sequence
  Synthetic Oligonucleotide
ttcttgtcaatctgcactat

SEQ ID NO: 7 - DNA, 177 bp, Artificial Sequence
  Synthetic Polynucleotide
cgtccatgcactgactacaagacaaatacctcactatgtttgttagtgcttggtaatataaaaccttttcttttatgagaaagttcaccgaaaataattttctatttgtggcataactagtatcgaagtatatagtgcagattgacaagaatttaattttgcagttgggcacatgaa

SEQ ID NO: 8 - protein, 669 aa, Solanum lycopersicum
MYSTVFYTSVHPSTSVLSRKQLPLLISKDFSAELYHSLPCRSLENGHINKVKGVKVKATIAEAPVTPTEKTDSGANGDLKVPQKKLKVLVAGGGIGGLVFALAAKKRGFDVLVFERDLSAIRGEGQYRGPIQIQSNALAALKAIDLDVAEDIMNAGCITGQRINGLVDGISGNWYCKFDTFTPAVERGLPVTRVISRMTLQQILARAVGEEIIMNESNVVDFEDDGEKVTVVLENGQRFTGDLLVGADGIRSKVRTNLFGPSEATYSGYTCYTGIADFVPADIDTVGYRVFLGHKQYFVSSDVGGGKMQWYAFYNEPAGGADAPNGKKERLLKIFGGWCDNVIDLLVATDEDAILRRDIYDRPPTFSWGRGRVTLLGDSVHAMQPNLGQGGCMAIEDSYQLALELEKACSRSAESGSPVDIISSLRSYESARKLRVGVIHGLARMAAIMASTYKAYLGVGLGPLSFLTQYRIPHPGRVGGRVFIDLGMPLMLSWVLGGNGDKLEGRIKHCRLSEKANDQLRKWFEDDDALERATDAEWLLLPAGNGSSGLEAIVLSRDEDVPCTVGSISHTNIPGKSIVLPLPQVSEMHARISCKDGAFFVTDLRSEHGTWVTDNEGRRYRTSPNFPTRFHPSDVIEFGSDKAAFRVKAMKFPLKTSERKEEREAVEAA

SEQ ID NO: 9 - DNA, 2009 bp, Solanum lycopersicum
atgtattcaactgttttttacacttcagtgcatccttccacttcagttttatcaagaaagcagctacctttattgatttccaaggacttttctgcagagttatatcattctttaccttgtaggagcttggaaaatgggcatatcaacaaggttaaaggagtaaaagtaaaagcaacaattgctgaagctccagttactcctacagagaagactgactctggggctaacggtgatttgaaggttccacagaagaagttgaaagtacttgttgcgggtggtgggattggtgggttagtgtttgctttggcagcaaagaaaagggggtttgatgtattggtgtttgagagggatttaagtgctatcagaggagagggacaatatagaggtccaatacagatacagagcaatgcattggctgctttaaaagcaattgatttggatgttgctgaagacattatgaatgctggctgcatcactggtcaaaggattaatggcttggttgatggtatttctggcaactggtattgcaagtttgatacgttcactccagcagtggaacgtggacttccagtgacaagagttatcagtcgcatgactttgcagcagatccttgcacgtgctgttggtgaggagataattatgaatgaaagtaatgttgtagactttgaggatgatggagagaaggttactgtggttcttgagaatggacaacgatttacaggtgatcttctggttggtgctgatggcataagatctaaggtacggactaatttattcggacccagtgaagctacttactctggctacacttgttatactggaattgcagatttcgttccagctgatattgatacagttgggtaccgcgtctttttgggccacaaacagtactttgtttcttcagatgtggggggaggcaagatgcagtggtatgcattttacaatgaaccagctggtggtgcggatgccccaaacggtaaaaaggaaagattgcttaaaatatttgggggatggtgtgacaatgttatagacctattagttgccacagatgaagatgcaattcttcgtcgtgacatctatgatagaccgccaacctttagttggggaagaggtcgtgttacattgcttggggactccgtccatgccatgcagcctaatttgggtcaagggggatgcatggccatagaggatagctatcaactagcactggaacttgaaaaagcatgcagtcgaagtgcagagtctggaagccctgtggatatcatctcatctttaaggagctatgaaagtgctagaaaacttcgagttggagtcatccatggactggctagaatggctgcaatcatggcatctacttacaaggcttatcttggcgtcggacttggtccactatcatttttgacgcagtatagaataccacatcctggaagagttggtggaagagtttttattgacttgggaatgcctctgatgttaagttgggttctaggaggcaatggggacaagcttgaaggcagaataaaacattgcaggctatctgagaaagcaaatgaccaattgagaaaatggtttgaagatgatgatgctttagagcgtgctactgatgcagagtggttacttttacctgcggggaatggctcttctggtttagaagctattgttttaagcagagatgaggatgtcccttgcactgtcgggtccatctcacatacaaatattcctggaaaatcaatagttttacctttgccacaggtatctgaaatgcacgcccggatatcatgcaaagacggagcattttttgtaactgatttacgaagtgaacatggtacctgggttacagataatgaaggcagaagatatcggacgtctccaaacttccctacacgttttcatccatcagatgttatcgaatttggttctgataaggcagcatttcgtgttaaagcaatgaaatttccactaaaaacttctgaaaggaaggaagagcgcgaagcagtggaggcagcgta

SEQ ID NO: 10 - DNA, 20 bp, Artificial Sequence
  Synthetic Oligonucleotide
aacccacaaatcccactttc

SEQ ID NO: 11 - DNA, 20 bp, Artificial Sequence
  Synthetic Oligonucleotide
ttctcttcggacaagcacac

SEQ ID NO: 12 - DNA, 800 bp, Artificial Sequence
  Synthetic Polynucleotide
aacccacaaatcccactttctttggcctaattaacaattttttcaataaatcttcttttcactgaatcatacatacattggaagaaaaactcaaatcttcttttcttgaactggggttcaattaaaagatgtattcaaatgtattcaactgttttttacacttcagtgcatccttccacttcagttttatcaagaaagcagctacctttattgatttccaaggacttttctgcagagttatatcattctttaccttgtaggagcttggaaaatgggcatatcaacaaggttaaaggagtaaaagtaaaagcaacaattgctgaagctccagttactcctacagagaagactgactctggggctaacggtgatttgaaggttccacagaagaagttgaaagtacttgttgcgggtggtgggattggtgggttagtgtttgctttggcagcaaagaaaagggggtttgatgtattggtgtttgagagggatttaagtgctatcagaggagagggacaatatagaggtccaatacagatacagagcaatgcattggctgctttaaaagcaattgatttggatgttgctgaagacattatgaatgctggctgcatcactggtcaaaggattaatggcttggttgatggtatttctggcaactggtaaattcacatcactatgatttctttgtgctaattaggtgcttgtttatgctttactctgatatgttttacttgagtcgacatcaaaagttagggataccaccctcctcagaacccactcgtgtgcttgtccgaagagaa

SEQ ID NO: 13 - DNA, 59 bp, Artificial Sequence
  Synthetic Oligonucleotide
gtctctatttttttttgggcatctttgcagctagtaaatttttctctctcttccgaatt

SEQ ID NO: 14 - DNA, 28 bp, Artificial Sequence
  Synthetic Oligonucleotide
actgagtaactccctctagtcagaaaga

SEQ ID NO: 15 - DNA, 411 bp, Artificial Sequence
  Synthetic Polynucleotide
gtctctatttttttttgggcatctttgcagctagtaaatttttctctctcttccgaattctttttgtttgtctgtaagctgtcaagttctgatttattttcaatcaactcttccaattgatgcttccttaatatatataacatgtcatttcagacatcacacaagcaatagcctatcagattacagattagggtttcaggttacaccaaaagagtcaagttgctttttctttttcaaagttaggattttttttttctttggtgaagcgaagggatcgaactctcagtaataaagtgaaagttaaaatagttctttggaaaaaaagagaaactttttcaactcgtcataattgaattaggattatttttacaagcaaagcaatttctttctgactagagggagttactcagt

SEQ ID NO: 16 - DNA, 20 bp, Artificial Sequence
  Synthetic Oligonucleotide
ttcttcggattgtccatggt

SEQ ID NO: 17 - DNA, 20 bp, Artificial Sequence
  Synthetic Oligonucleotide
caccaatgctatgtgccaaa

SEQ ID NO: 18 - DNA, 697 bp, Artificial Sequence
  Synthetic Polynucleotide
ttcttcggattgtccatggtataattgatcagagttggcatctttgatgcatacaatggttgcctaagaatgctggtgcaccctttgcagatccatcgtgccagaagattttatgagatcgttgtaccaagttataccatatacgatgttgaatgtcccgaccattcatttcgcaagttcacggatgacggtctatattttgtaagtttcagccgaaaccatcaggatctggttgtttatagaccaacatggctgacattttcctgcaaagaagaagattgtgatactcatgatcttcctttgaaagctagaaagtttgagagcttcttcacacagttgtacagtgttactcttgcttctagtggggaacttatatgcaaagatttctttctctatatggagagcaaccaatttggactctttgcaacttcaactgcacaaattcatgatgcacctcctactggaggggcaattcagggagtcccttcagttgaaaaaataactttccaccttttgaggtgtgtgatcagtctcaactattaatttcgtgagcacttaacatgatgttttggtctctgaagttatcttggttgcttttgttgtggttggtttcattaacttaggttggtggatggagctatacttgacgaaagggttttccacaatgattatgttaatttggcacatagcattggtg

SEQ ID NO: 19 - DNA, 22 bp, Artificial Sequence
  Synthetic Oligonucleotide
ctcgatttcaatttccttcagc

SEQ ID NO: 20 - DNA, 25 bp, Artificial Sequence
  Synthetic Oligonucleotide
ccatcctaaacttgatgttcttgtc

SEQ ID NO: 21 - DNA, 20 bp, Artificial Sequence
  Synthetic Oligonucleotide
atcttgtggcagctgtgatg

SEQ ID NO: 22 - DNA, 22 bp, Artificial Sequence
  Synthetic Oligonucleotide
tgacctccaactttcataatgc

SEQ ID NO: 23 - DNA, 21 bp, Artificial Sequence
  Synthetic Oligonucleotide
atggcggtcggactattggcc

SEQ ID NO: 24 - DNA, 20 bp, Artificial Sequence
  Synthetic Oligonucleotide
tgatccaaaaattggtcctc

SEQ ID NO: 25 - DNA, 22 bp, Artificial Sequence
  Synthetic Oligonucleotide
ctatccttcgcaagacccttcc

SEQ ID NO: 26 - DNA, 20 bp, Artificial Sequence
  Synthetic Oligonucleotide
gcaaaacttttcgagccaac

SEQ ID NO: 27 - DNA, 32 bp, Artificial Sequence
  Synthetic Oligonucleotide
agccactagttgcccgcgtggccgagaaattg

SEQ ID NO: 28 - DNA, 31 bp, Artificial Sequence
  Synthetic Oligonucleotide
gtcgctcgagctgatccaaaaattggtcctc

SEQ ID NO: 29 - DNA, 21 bp, Artificial Sequence
  Synthetic Oligonucleotide
ccacaatccacaatccacttc